(19) United States
(12) Patent Application Publication
(10) Pub. No.: US 2026/0207222 A1
(43) Pub. Date: Jul. 23, 2026

(54) STEERABLE TUBES

(71) Applicant: Edwards Lifesciences Corporation, Irvine, CA (US)

(72) Inventors: Peleg Harel, Haifa (IL); Eran Grosu, Kfar-Saba (IL); Elazar Levi Schwarcz, Netanya (IL); Eitan Atias, Tel Aviv (IL); Ofir Witzman, Harish (IL); David Maimon, Atlit (IL); Halit Yaakobovich, Netanya (IL); Dmitry Pinhasov, Yokneam Illit (IL); Yaron Herman, Givat Ada (IL)

(73) Assignee: Edwards Lifesciences Corporation, Irvine, CA (US)

(21) Appl. No.: 19/562,590

(22) Filed: Mar. 10, 2026

Related U.S. Application Data
(63) Continuation-in-part of application No. PCT/US2024/048278, filed on Sep. 25, 2024.
(63) Continuation-in-part of application No. PCT/US2024/048287, filed on Sep. 25, 2024.
(63) Continuation-in-part of application No. PCT/US2024/047172, filed on Sep. 18, 2024.
(60) Provisional application No. 63/612,934, filed on Dec. 20, 2023.
(60) Provisional application No. 63/540,990, filed on Sep. 28, 2023.
(60) Provisional application No. 63/540,757, filed on Sep. 27, 2023.
(60) Provisional application No. 63/583,915, filed on Sep. 20, 2023.

(51) Int. Cl. A61B 17/34; A61B 17/00

(52) U.S. Cl. CPC A61B 17/3403; A61B 17/3417; A61B 2017/00243; A61B 2017/00318; A61B 2017/00557; A61B 2017/3456

(57) ABSTRACT

The present disclosure relates to steerable tubes that can be used for orienting a needle piercing tip towards a target tissue, such as a host leaflet within which a guest prosthetic valve can be expanded. In an example, a steerable tube comprises two longitudinal slots defining a first tube portion and a second tube portion, and a slotted section that includes a plurality of pairs of circumferential slots axially spaced from each other along the second tube portion. The first tube portion is configured, when proximally pulled relative to the second tube portion, to transition the slotted section from a first or unbent state to a second or bent state. In some examples, the steerable tube is a steerable needle terminating at a piercing tip. In some examples, the delivery apparatus further comprises a needle terminating at a piercing tip, which is axially movable through or around the steerable tube.

### CROSS-REFERENCE TO RELATED APPLICATIONS

This application is a continuation-in-part of International Application No. PCT/US2024/048287, filed September 25, 2024, which claims the benefit of U.S. Provisional Application No. 63/540,990, filed September 28, 2023. This application is also a continuation-in-part of International Application No. PCT/US2024/048278, filed September 25, 2024, which claims the benefit of U.S. Provisional Application No. 63/540,757, filed September 27, 2023, and U.S. Provisional Application No. 63/612,934, filed December 20, 2023. This application is also a continuation-in-part of International Application No. PCT/US2024/047172, filed September 18, 2024, which claims the benefit of U.S. Provisional Application No. 63/583,915, filed September 20, 2023, each of the foregoing which is incorporated by reference herein in its entirety.

### FIELD

The present disclosure relates to delivery apparatuses that include a steerable tube, configured to steer a piercing needle tip towards a target tissue in order to form an opening in the target tissue, and to methods and devices for puncturing through a target tissue that can be a leaflet of an existing valvular structure, in a manner that can modify existing valvular structures (for example, leaflets of a native heart valve or previously-implanted prosthetic valve) prior to implantation of a guest prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient’s vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve ("guest valve") is delivered to the heart in a crimped state, as described above for the "native" TAVR. The guest valve is positioned within the previously implanted prosthetic valve ("host valve") and then expanded to its functional size. The host valve in a ViV TAVR procedure can be a surgically implanted prosthetic valve or a transcatheter prosthetic valve. The term "host valve" is also used herein to refer to the native aortic valve in a native TAVR procedure.

### SUMMARY

Needles or other cutting tools can be utilized for piercing existing leaflets to form an opening that modifies the existing valvular structure, after which a guest prosthetic valve can be implanted in the modified valvular structure, mitigating the risk of coronary ostial obstruction. Approximation of a needle towards an existing leaflet in the vicinity of a coronary ostium is challenging, as the delivery apparatus can be oriented to direct the needle towards an internal surface of the existing valve instead of towards the surface of the target leaflet itself, posing a risk of puncturing or otherwise damaging surfaces adjacent the target leaflet when the needle is advanced for penetration.

According to some aspects of the disclosure, there is provided a delivery apparatus comprising a handle and a steerable tube extending distally from the handle and defining a steerable tube lumen. The steerable tube comprises two longitudinal slots circumferentially spaced from each other and defining a first tube portion therebetween and a second tube portion. The steerable tube further comprises a slotted section, and a backbone opposite to the first tube portion. The slotted section comprises a plurality of pairs of circumferential slots, axially spaced from each other along the second tube portion. Each circumferential slot of the plurality of pairs of circumferential slots extends between a slot free end at one of the two longitudinal slots and a slot backbone end. The backbone opposite to the first tube portion is defined between the slot backbone ends of the plurality of pairs of circumferential slots. The delivery apparatus further comprises a steerable tube distal portion, extending from longitudinal slot distal ends of the two longitudinal slots to a steerable tube distal edge. Axial movement of the first tube portion and/or the second tube portion relative to each other is configured to change the slotted section between a first state and a second state, such that the radius of curvature of the slotted section in the second state is different than its radius of curvature in the first state.

According to some aspects of the disclosure, there is provided a method comprising advancing a delivery apparatus comprising a steerable tube, over a guidewire, to a host valvular structure. The steerable tube comprises two longitudinal slots defining a first tube portion and a second tube portion, a steerable tube distal portion extending distally from the longitudinal slots, and a slotted section. The slotted section comprises a plurality of pairs of circumferential slots disposed along the second tube portion and defining a backbone opposite to the first tube portion. The method further comprises bending the slotted section by axially moving the first tube portion and the second tube portion relative to each other. The method further comprises forming, with a piercing tip of the delivery apparatus, a pilot puncture within a host leaflet of the host valvular structure.

According to some aspects of the disclosure, there is provided a leaflet piercing device comprising a tube defining a lumen extending along a longitudinal axis. The tube comprises a first longitudinal slot, a second longitudinal slot, and a first tube portion longitudinally extending between the first and second longitudinal slots. The tube further comprises a second tube portion opposite of the first tube portion and comprising a backbone, and a plurality of circumferential slots, which circumferentially extend between the first and second longitudinal slots and the backbone. The tube further comprises a piercing distal tip, wherein a relative longitudinal movement of the first tube portion relative to the second tube portion is configured to bend the tube such that the piercing distal tip is offset from the longitudinal axis.

According to some aspects of the disclosure, there is provided a method of forming a puncture in a host leaflet, the method comprising advancing a tube, over a guidewire, to a host valvular structure. The tube comprises a slotted section comprising a first longitudinal slot, a second longitudinal slot, a backbone, and a plurality of circumferential slots formed between a first longitudinal slot and a second longitudinal slot. The tube further comprises a first tube portion extending between the first longitudinal slot and the second longitudinal slot, a second tube portion, and a distal piercing tip. The method further comprises moving the first tube portion relative to the second tube portion, to change the slotted section between a first configuration and a second configuration, and advancing the tube to form a puncture within a host leaflet with the distal piercing tip.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures

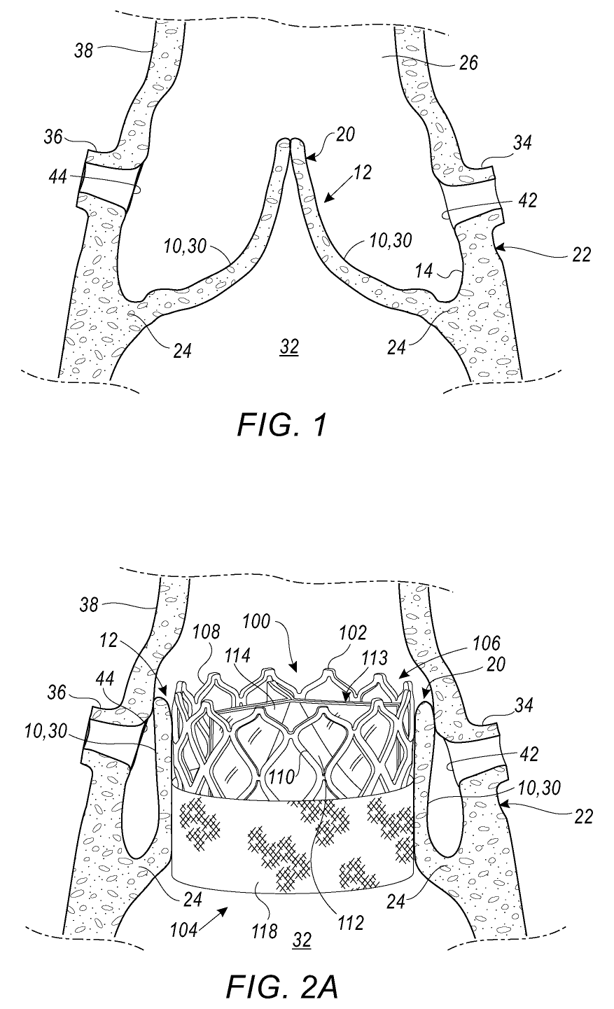

FIG. 1 is a cross-sectional view of a native aortic valve.

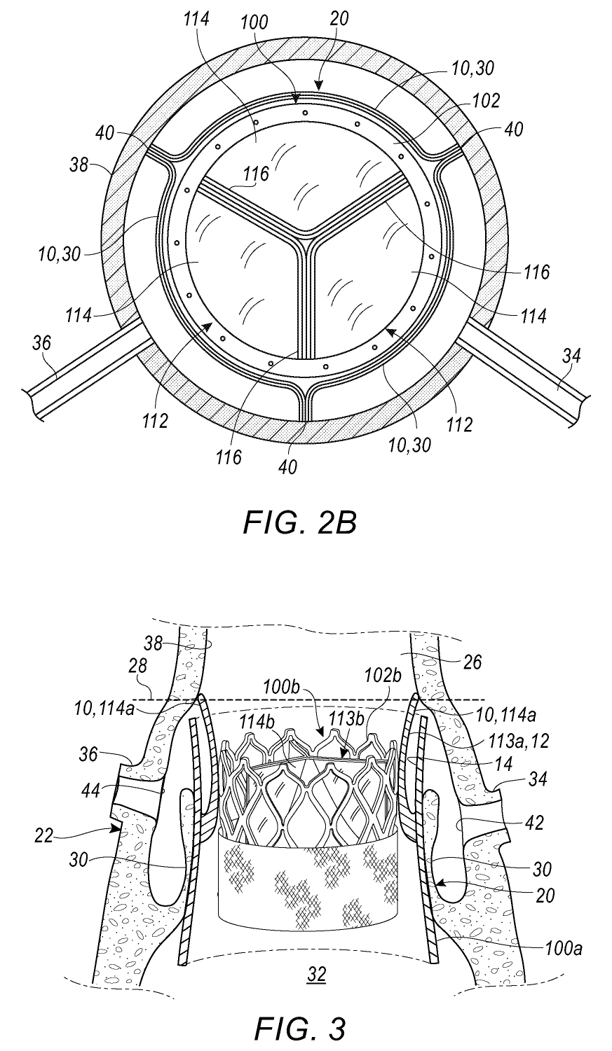

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

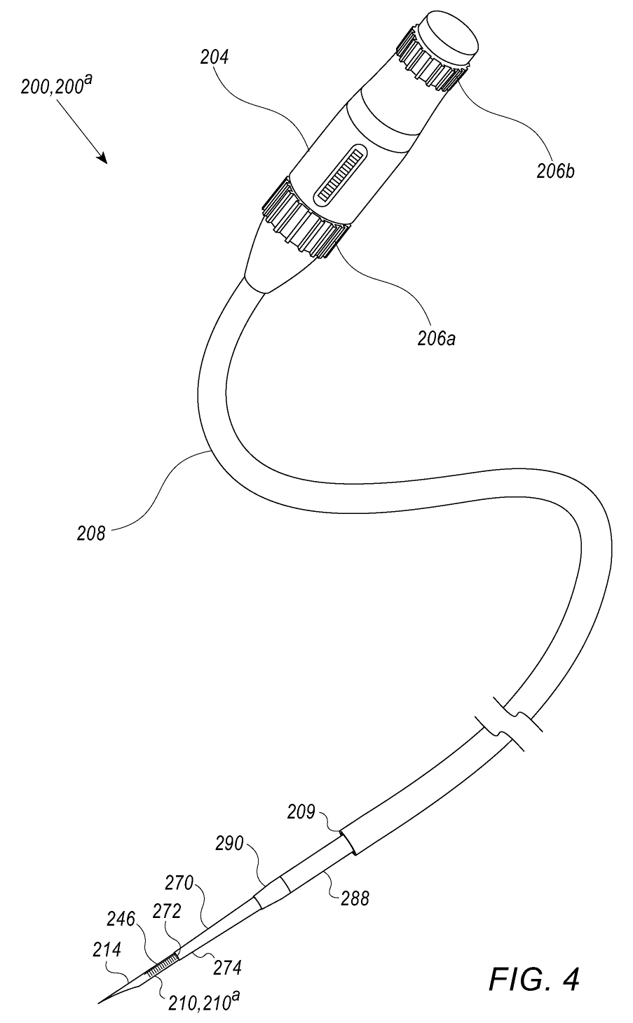

FIG. 4 shows an exemplary delivery apparatus adapted to deliver a low-profile steerable tube.

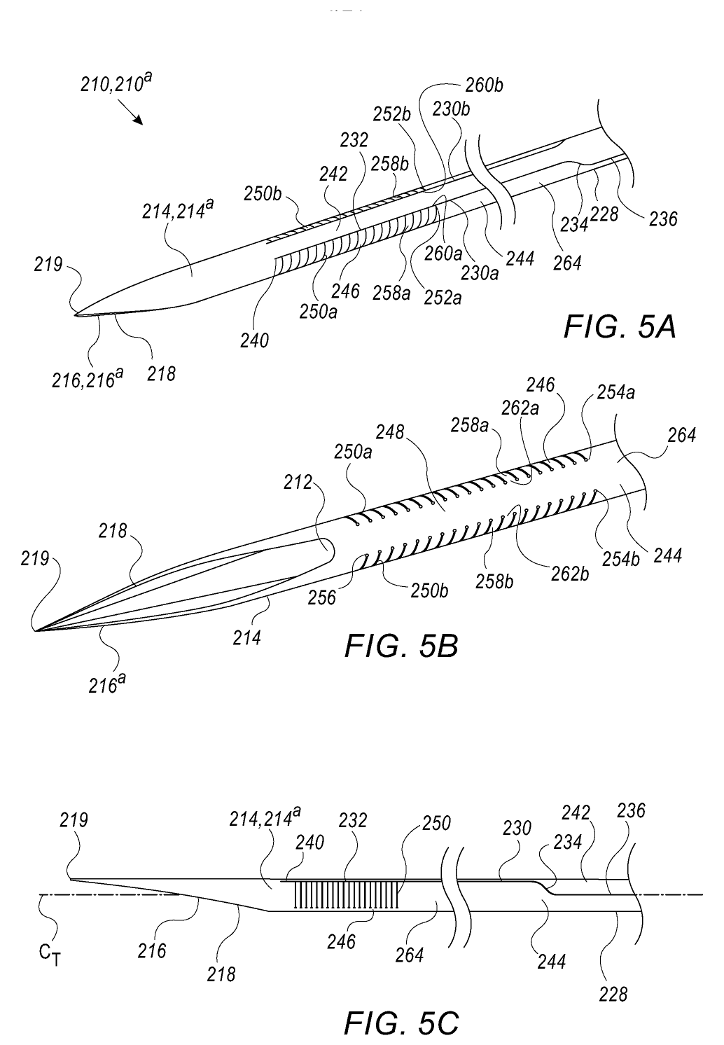

FIG. 5A is a perspective view of an exemplary steerable tube implemented as a steerable needle.

FIG. 5B is a perspective view of a distal region of the steerable tube of FIG. 5A from another view angle.

FIG. 5C is a side view of the steerable tube of FIGS. 5A-5B.

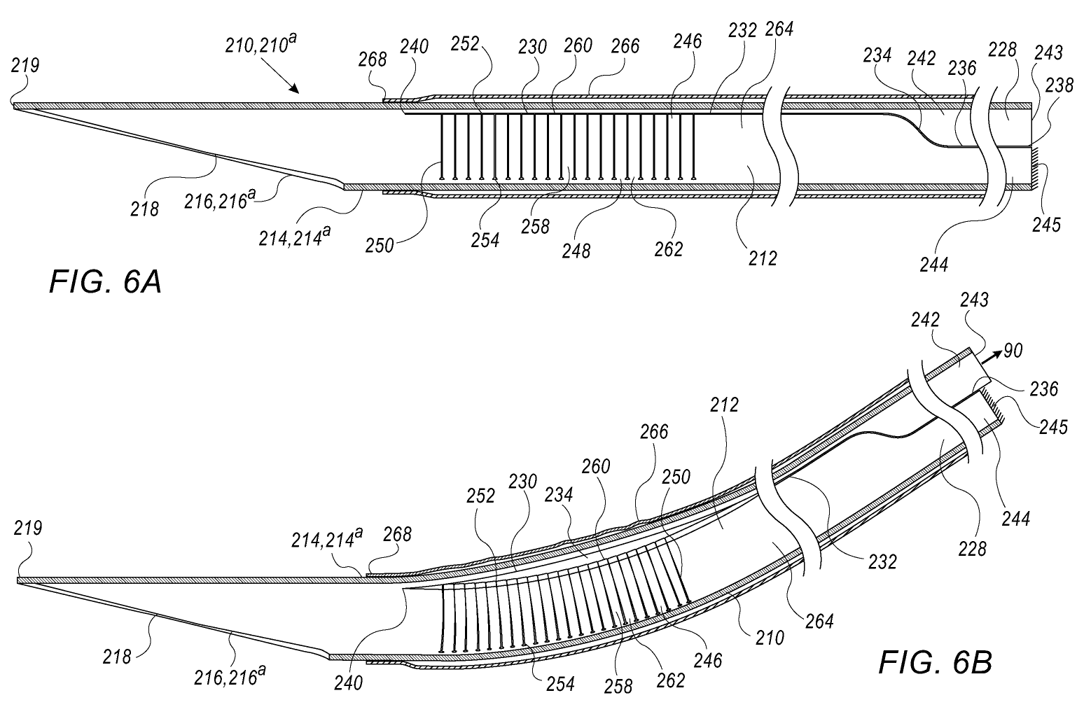

FIG. 6A is a cross-sectional view of the steerable tube of FIGS. 5A-5C, covered by a sleeve, in a first or unbent state of the steerable tube.

FIG. 6B is a cross-sectional view of the steerable tube of FIG. 6A, in a second or bent state thereof.

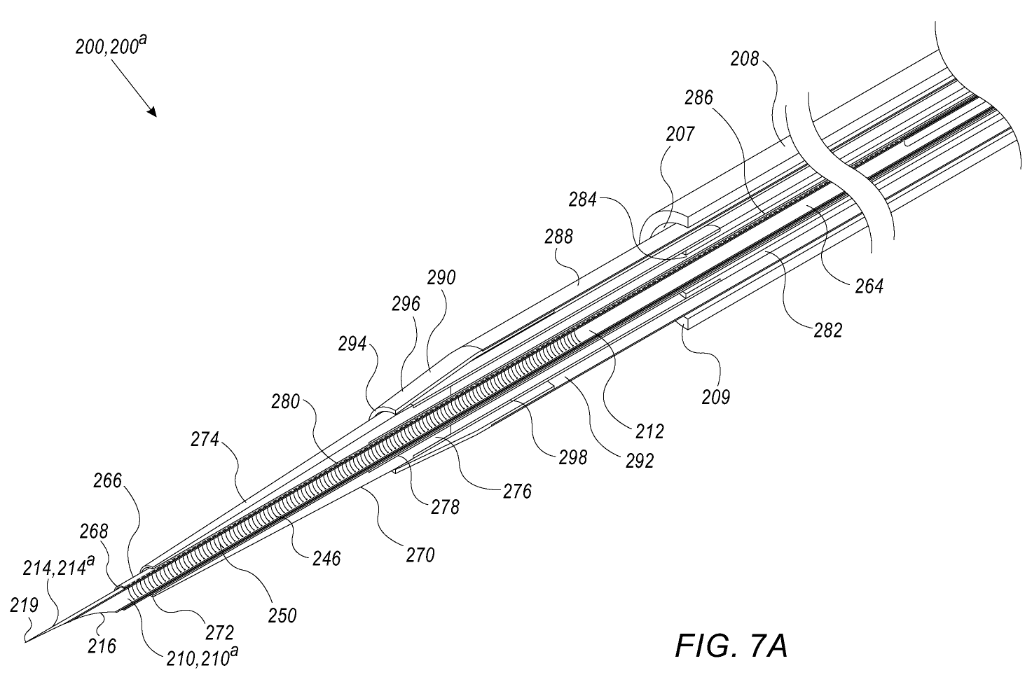

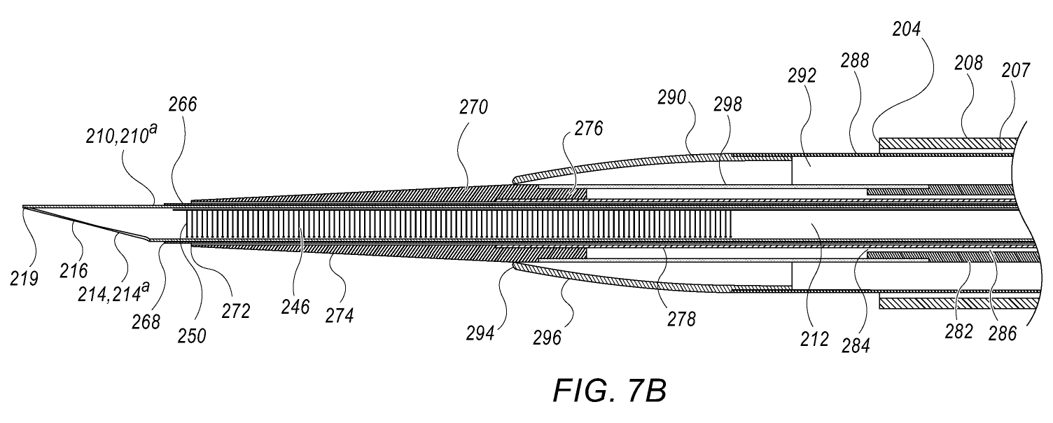

FIG. 7A is a perspective view of a distal region of an exemplary delivery apparatus, comprising a steerable needle.

FIG. 7B is a cross-sectional view of a distal region of the delivery apparatus of FIG. 7A.

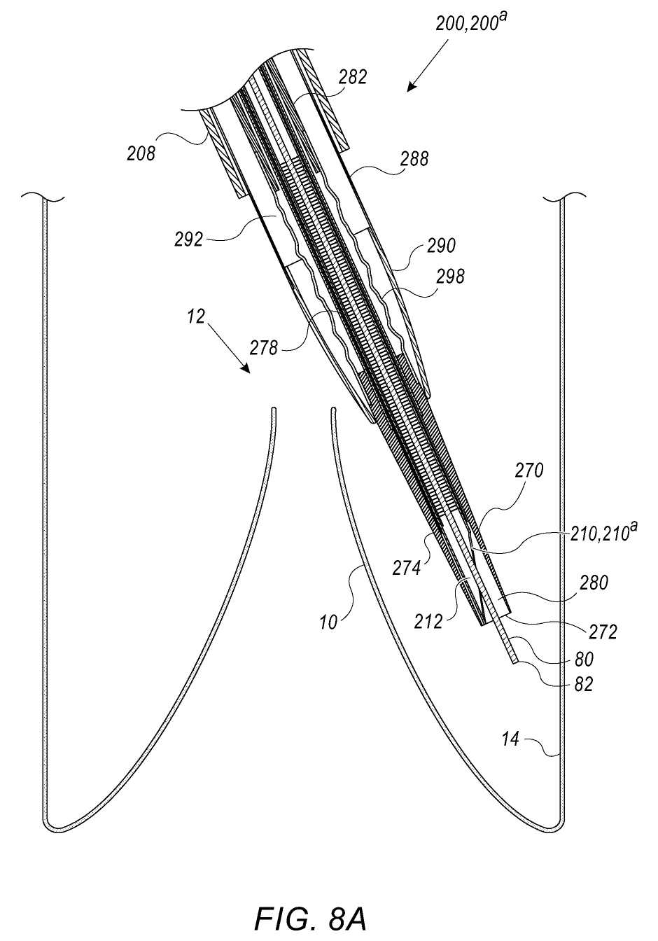

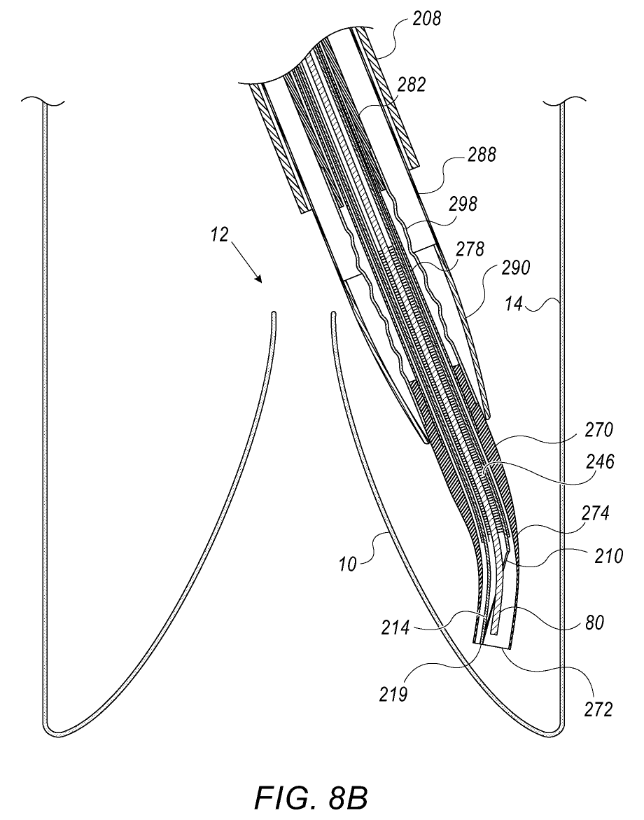

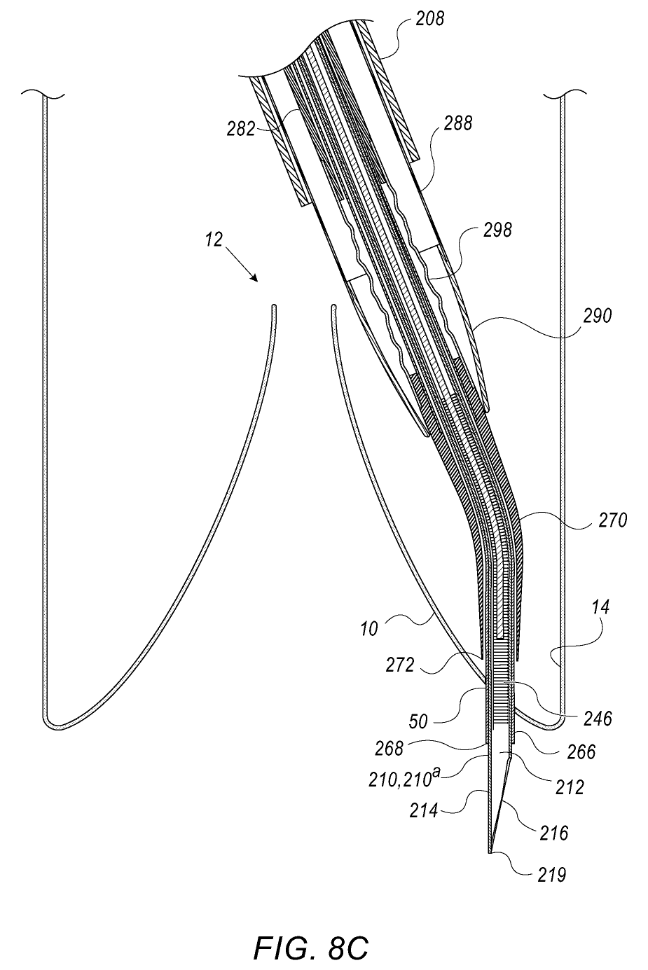

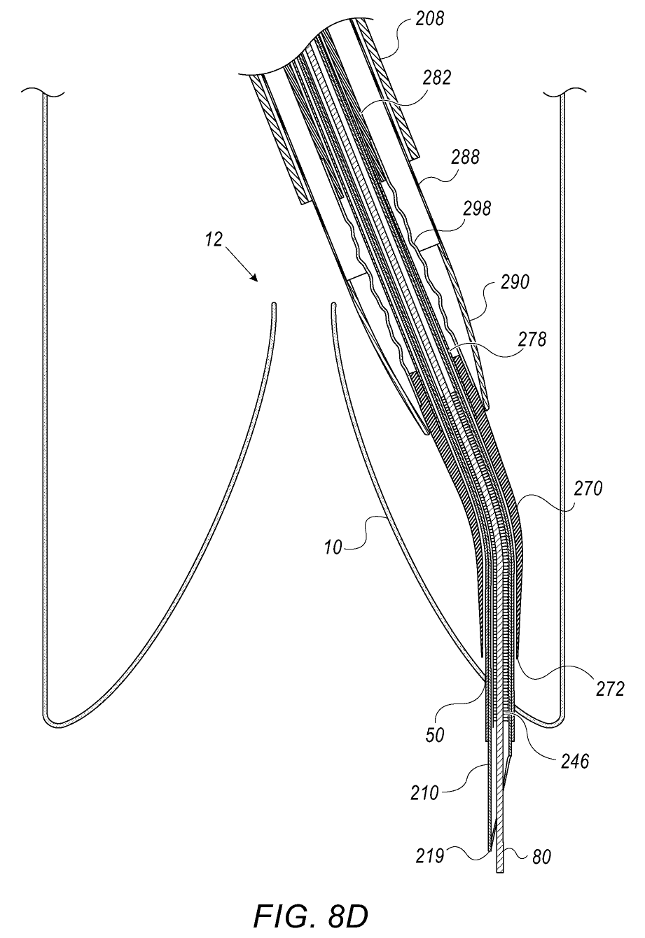

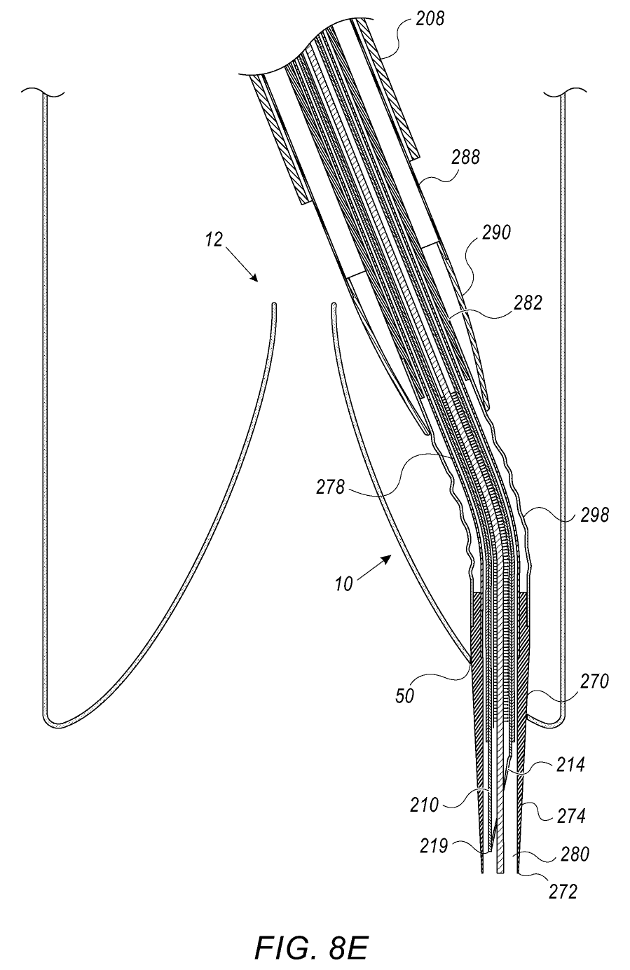

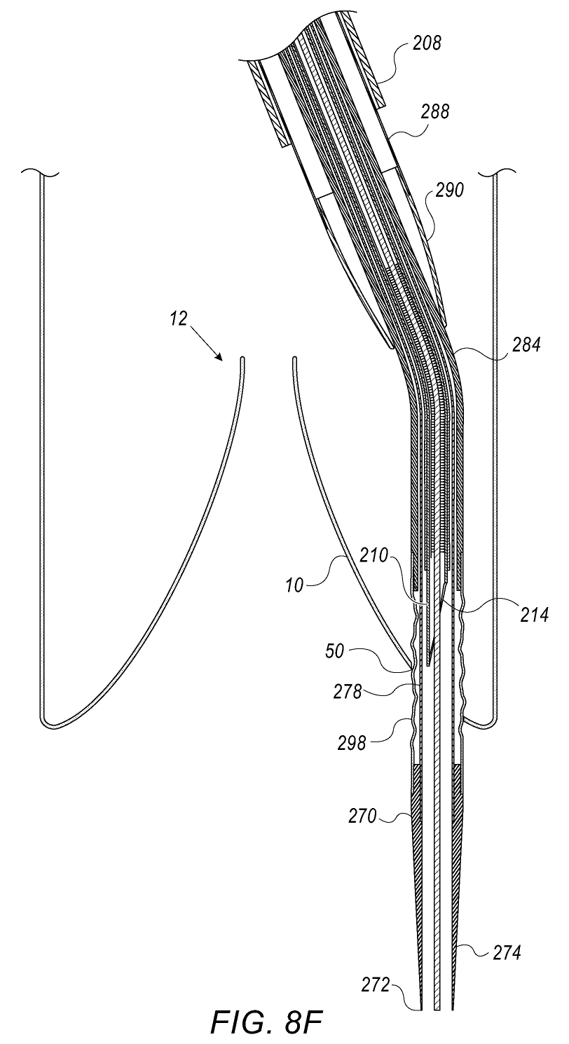

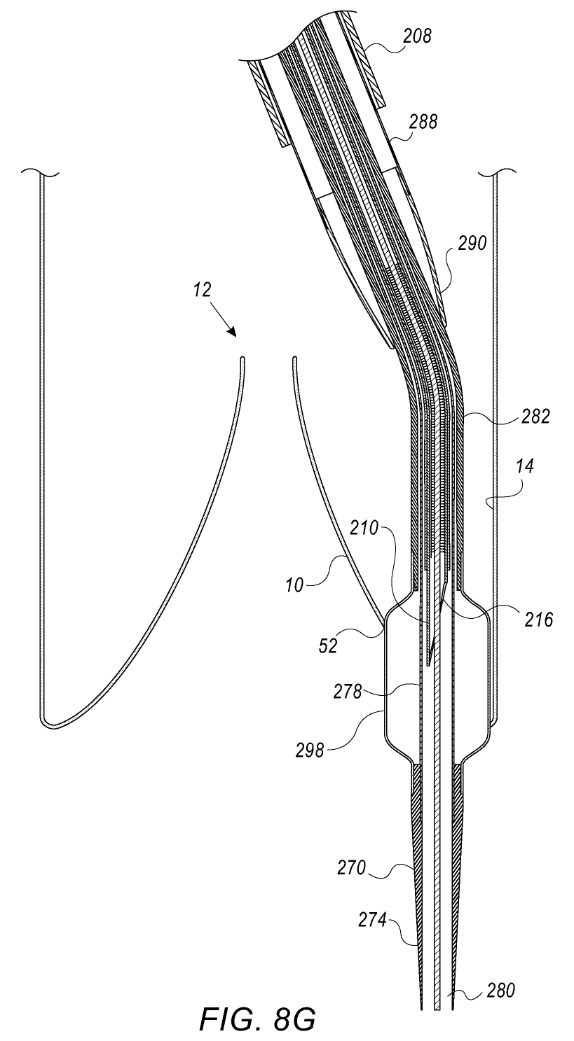

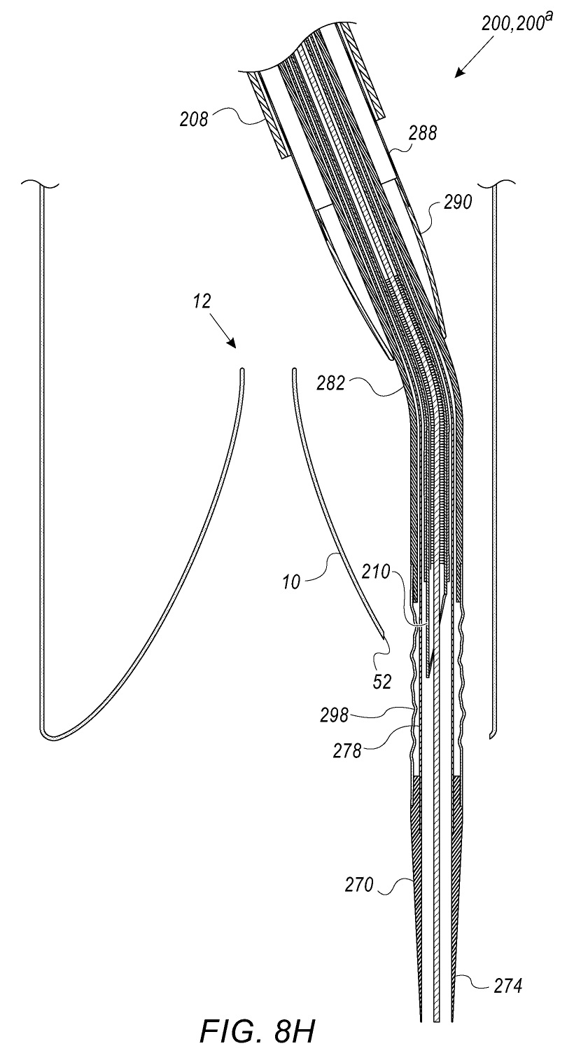

FIGS. 8A-8H illustrate steps in a method for utilizing an exemplary delivery apparatus for forming an opening within a host leaflet.

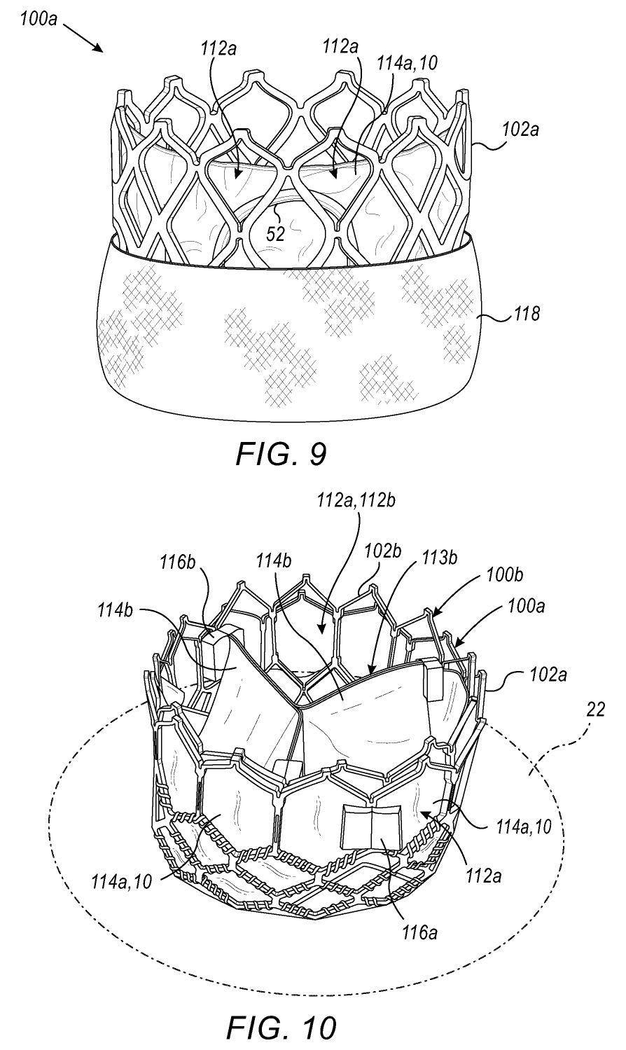

FIG. 9 is a perspective view of a host prosthetic valve subsequent to forming a leaflet opening thereof.

FIG. 10 is a perspective view of a guest prosthetic valve expanded within a leaflet opening of a host prosthetic valve.

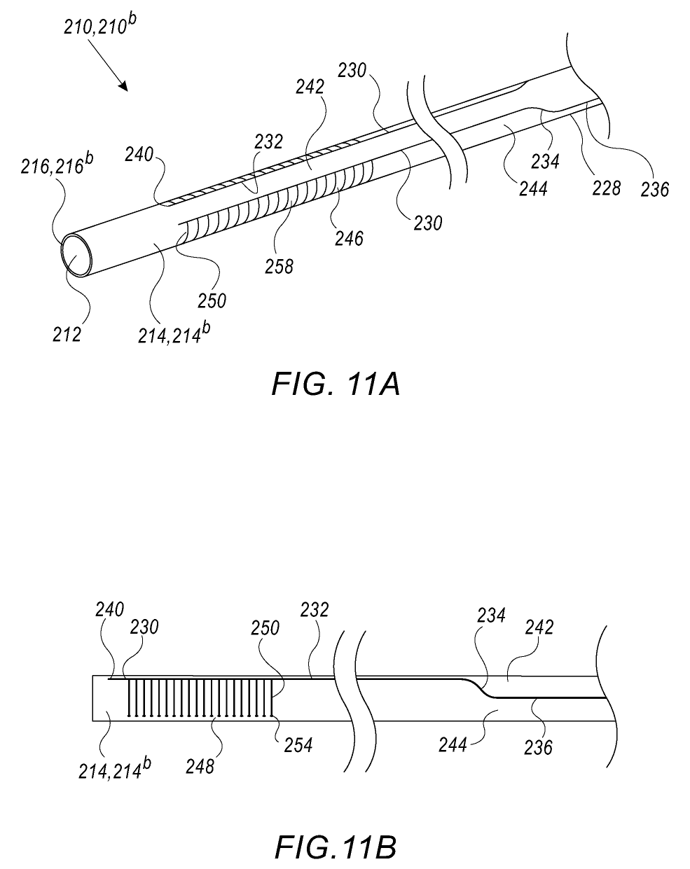

FIG. 11A is a perspective view of an exemplary steerable tube devoid of a piercing tip.

FIG. 11B is a side view of the steerable tube of FIG. 11A.

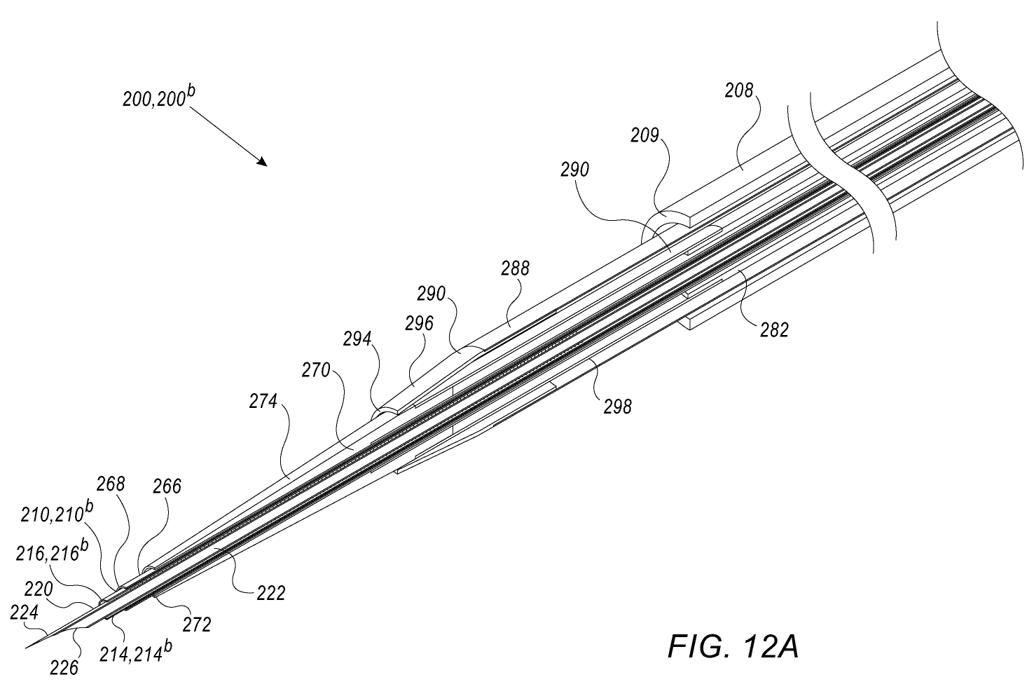

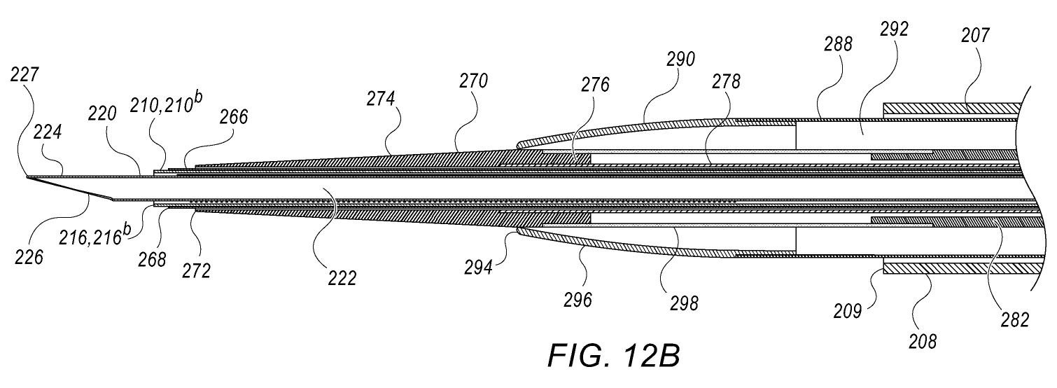

FIG. 12A is a perspective view of a distal region of an exemplary delivery apparatus, comprising a needle disposed inside a lumen of the steerable tube.

FIG. 12B is a cross-sectional view of a distal region of the delivery apparatus of FIG. 12A.

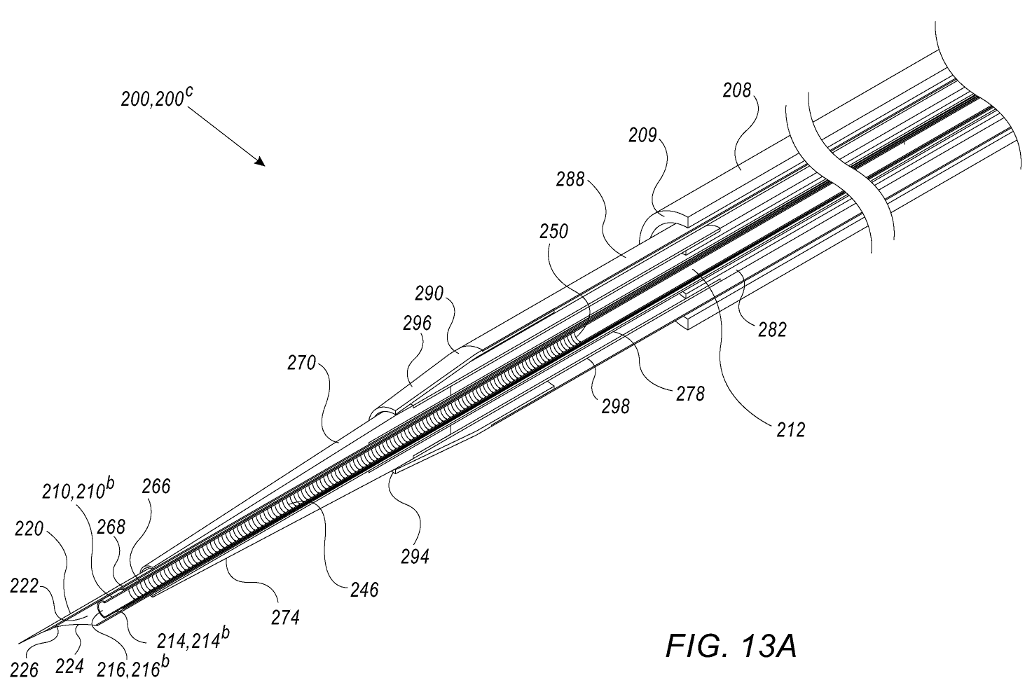

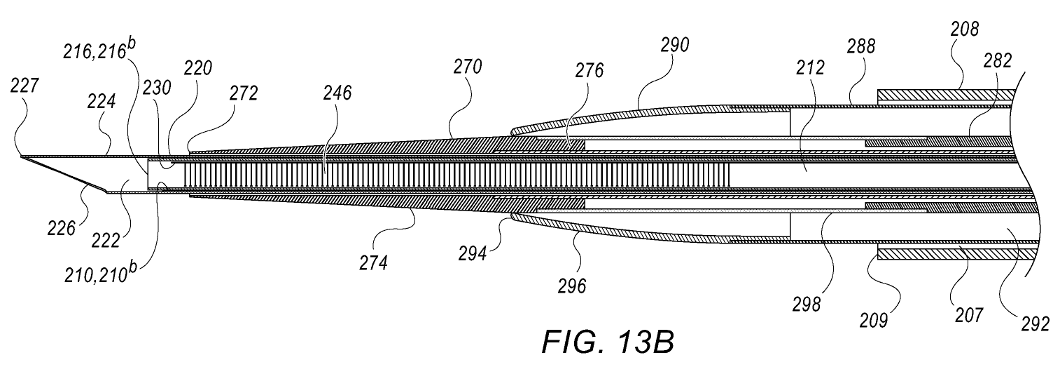

FIG. 13A is a perspective view of a distal region of an exemplary delivery apparatus, comprising a needle disposed around the steerable tube.

FIG. 13B is a cross-sectional view of a distal region of the delivery apparatus of FIG. 13A.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like "provide" or "achieve" to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms "a", "an", and "the" include the plural forms unless the context clearly dictates otherwise. Additionally, the terms "have" or "includes" means "comprises". Further, the terms "coupled", "connected", and "attached", as used herein, are interchangeable and generally mean physically, mechanically, chemically, magnetically, and/or electrically coupled or linked and does not exclude the presence of intermediate elements between the coupled or associated items absent specific contrary language. As used herein, "and/or" means "and" or "or", as well as "and" and "or".

Directions and other relative references may be used to facilitate discussion of the drawings and principles herein, but are not intended to be limiting. For example, certain terms may be used such as "inner", "outer", "upper", "lower", "inside", "outside", "top", "bottom", "interior", "exterior", "left", right", and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an "upper" part can become a "lower" part simply by turning the object over. Nevertheless, it is still the same part and the object remains the same.

The term "plurality" or "plural" when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms "proximal" and "distal" are defined relative to the use position of a delivery apparatus. In general, the end of the delivery apparatus closest to the user of the apparatus is the proximal end, and the end of the delivery apparatus farthest from the user (for example, the end that is inserted into a patient’s body) is the distal end. The term "proximal" when used with two spatially separated positions or parts of an object can be understood to mean closer to or oriented towards the proximal end of the delivery apparatus. The term "distal" when used with two spatially separated positions or parts of an object can be understood to mean closer to or oriented towards the distal end of the delivery apparatus. The terms "longitudinal" and "axial" are interchangeable, and refer to an axis extending in the proximal and distal directions, unless otherwise expressly defined.

The terms "axial direction", "radial direction", and "circumferential direction" have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms "integrally formed" and "unitary" refer to a construction that does not include any welds, fasteners, or other means for securing separately formed pieces of material to each other.

As used herein, operations that occur "simultaneously" or "concurrently" occur generally at the same time as one another, although delays in the occurrence of operation relative to the other due to, for example, spacing between components, are expressly within the scope of the above terms, absent specific contrary language.

As used herein, terms such as "first", "second", and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term "substantially" means the listed value and/or property and any value and/or property that is at least 75% of the listed value and/or property. Equivalently, the term "substantially" means the listed value and/or property and any value and/or property that differs from the listed value and/or property by at most 25%. For example, "at least substantially parallel" refers to directions that are fully parallel, and to directions that diverge by up to 22.5 degrees.

In the present disclosure, a reference numeral that includes an alphabetic label (for example, "a", "b", "c", etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient’s heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

FIGS. 2A-2B show an exemplary prosthetic valve 100 that can be implanted in a native heart valve, such as the native aortic valve 20 of FIG. 1. The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Patent No. 10,603,165, International Application No. PCT/US2021/052745 and U.S. Provisional Application Nos. 63/85,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term "outflow", as used herein, refers to a region of the prosthetic valve through which the blood flows through and out of the prosthetic valve 100.

The term "inflow", as used herein, refers to a region of the prosthetic valve through which the blood flows into the prosthetic valve 100.

In the context of the present application, the terms "lower" and "upper" are used interchangeably with the terms "inflow" and "outflow", respectively. Thus, for example, the lower end of the prosthetic valve is its inflow end and the upper end of the prosthetic valve is its outflow end.

In the context of the present application, the terms "lower" and "upper" are used interchangeably with the terms "distal to" and "proximal to", respectively. Thus, for example, a lowermost component can refer to a distal-most component, and an uppermost component can similarly refer to a proximal-most component.

The terms "longitudinal" and "axial", as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term "strut" encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in US Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Patent Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term "host valve" as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a "surgical valve") or a transcatheter heart valve. The term "guest valve", as used herein, refers to a prosthetic valve implanted in a host valve, which can be either a native heart valve or a previously implanted prosthetic valve. Similarly, the term "host leaflets 10", as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

When a guest prosthetic valve 100 is deployed inside a host valvular structure 12, it displaces the host leaflets 10 of the host valve radially outwards, towards and against a host interior surface 14, which can be the interior surface of the aortic wall 38 if the host valve is the native valve, or an interior surface of the frame 102a of a previously implanted prosthetic valve 100a serving as the host valve.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36.

FIG. 4 illustrates an exemplary delivery apparatus 200 adapted to deliver a low-profile steerable tube 210, which can be implemented as a steerable needle or can allow deployment of a needle therethrough, towards a host valvular structure 12, for modifying a host leaflet 10 thereof. According to some examples, the delivery apparatus 200 includes a handle 204 and outer shaft 208. A low-profile steerable tube 210 can extend through a lumen 209 of the outer shaft 208.

The outer shaft 208 and the steerable tube 210 can be configured to be axially movable relative to each other. For example, a distally oriented movement of the steerable tube 210 relative to the outer shaft 208, can expose a distal portion 214 of the steerable tube 210.

The proximal ends of the outer shaft 208 and the steerable tube 210 can be coupled to the handle 204. During delivery through the patient's vasculature, the handle 204 can be maneuvered by an operator (for example, a clinician or a surgeon) to axially advance or retract components of the delivery apparatus 200, such as the outer shaft 208 or any other component passing therethrough, including a steerable tube 210 which will be described in further detail below.

The handle 204 can include a steering mechanism configured to adjust the curvature of a distal slotted section 246 of the steerable tube 210. In the illustrated example, the handle 204 can include an adjustment member, such as the illustrated rotatable knob 206a, which in turn is operatively coupled to the proximal end portion of a first portion of steerable tube 210, which can be also referred to as a pullable portion of the steerable tube 210 and described in greater detail below. The first portion or pullable portion of first portion of steerable tube 210 can be a portion of the steerable tube 210 extending distally from the handle 204 and separated from a second portion of the steerable tube 210, also referred to as a non-pullable portion, along a length of the tube 210 extending from the handle 204 up to a distal portion of the tube 210, at which point both first and second portion of the tube 210 are joined, as will be described in greater detail below.

Rotating the knob 206a can increase or decrease the tension in the first portion of steerable tube 210, thereby adjusting the curvature of the distal slotted section 246 of the steerable tube 210. This can be accomplished by axially moving the first portion and/or the second portion of the tube 210 relative to each other, such as by pushing the second portion relative to the first portion, by pulling the first portion relative to the second portion, or both. In some examples, mechanisms for pulling a first or pullable portion of the tube 210 implemented in handle 204 can be similar to mechanisms implemented in a handle of a delivery apparatus for controlling the tension of a pull wire, as previously disclosed in U.S. Patent No. 9,339,384, which is incorporated by reference herein. The handle 204 can further include an adjustment mechanism including an adjustment member, such as the illustrated rotatable knob 206b. The adjustment mechanism can be configured to adjust the axial position of the steerable tube 210 relative to the outer shaft 208. The handle can include additional adjustment mechanisms controllable by additional knobs to maneuver additional components of the delivery apparatus 200, such as axial movement of other components and/or shafts that can be included in apparatus 200.

FIGS. 5A-5C show an exemplary steerable tube 210 from different view angles. A steerable tube 210 extends from a steerable tube proximal portion 228 to a steerable tube distal portion 214, and defines a steerable tube lumen 212 having a tube central axis C_{T}. Steerable tube 210 comprises a slotted section 246 extending proximally from the steerable tube distal portion 214. In some examples, a sleeve 266 can be disposed around steerable tube 210, as shown for example in FIGS. 6A-6B. Steerable tube 210 includes two longitudinal slots 230, each extending from a longitudinal slot proximal end 238 to a longitudinal slot distal end 240. The longitudinal slot distal ends 240 terminate at or proximal to the steerable tube distal portion 214, without extending into the steerable tube distal portion 214.

Two longitudinal slots, namely first longitudinal slot 230a and second longitudinal slot 230b, are illustrated in FIGS. 5A-5C, circumferentially dividing the steerable tube 210, along the section that includes slots 230, to a first tube portion 242 and a second tube portion 244. The first tube portion 242 proximally extends from the position of longitudinal slot distal ends 240 to a first portion proximal end 243, and the second tube portion 244 proximally extends from the position of longitudinal slot distal ends 240 to a second portion proximal end 245, as shown in FIG. 6A for example.

The slotted section 246 comprises a plurality of circumferential slots 250 extending around a circumference of the second tube portion 244, in a direction that can be substantially orthogonal to the tube central axis C_{T}. The slots 250 are configured to impart flexibility to the slotted section 246. Each slot 250 extends from a slot free end 252 defined at one of the longitudinal slots 230, to an opposite slot backbone end 254. The circumferential slots 250 are arranged in slot pairs, such that at each axial position of the slotted section 246 that includes slots 250, two circumferential slots 250a and 250b extend over opposite sides of the slotted section 246. Each pair of slots 250 is axially spaced from an adjacent pair of slots 250, and the slot pairs 250 define a backbone 248 between their slot backbone ends 254, such that the backbone 248 is positioned opposite to the first tube portion 242. Thus, the portion of the second tube portion 244 along the slotted section 246, which is not cut by slots 250, may be referred to as the backbone 248.

Thus, a pair of circumferential slots 250 is defined as a pair that include two circumferential slots 250, namely a first circumferential slot 250a and a second circumferential slot 250b, each extending over a different portion of a circumference of the steerable tube 210 and aligned with each other at the same axial position, while both circumferential slots 250 of the same pair are circumferentially spaced from each other, such as having their slot free ends 252 at the same axial position but circumferentially spaced from each other by the first tube portion 244, and having their slot backbone ends 254 at the same axial position but circumferentially spaced from each other by the backbone 248.

As shown, the plurality of circumferential slots 250 comprises a plurality of first circumferential slots 250a extending from first slot free ends 252a defined at first longitudinal slot 230a to first slot backbone ends 254a, and a plurality of second circumferential slots 250b extending from second slot free ends 252b defined at second longitudinal slot 230b to second slot backbone ends 254b, wherein each pair of slots 250 comprises a first circumferential slots 250a and a second circumferential slot 250b at the same axial position along slotted section 246, having their slot backbone ends 254a and 254b circumferentially spaced from each other by backbone 248.

In some examples, the longitudinal slots 230 can be substantially parallel to the tube central axis C_{T}, at least along the slotted section 246. In some examples, the first tube portion 242 can have a uniform width, defined between the longitudinal slots 230, at least along the slotted section 246. In some examples, the backbone 248 can have a uniform width, defined between the slot backbone ends 254, at least along the slotted section 246. In some examples, all of the slots 250 can have identical lengths, wherein a length of a circumferential slot is defined between its free end 252 and its backbone end 254.

In some examples, a slot backbone end 254 can include a strain relief region 256, defined to have a dimension in a direction that is parallel to the length of the slots 250, which is greater than the dimension of the slot 250 in the same direction, adjacent to the strain relief region 256. In the illustrated example, circular strain relief regions 256 are illustrated, though any other shape is contemplated, including T-shaped strain relief regions and the like.

It is to be understood that while the steerable tube 210 can include segments or sub-segments which are not necessarily steerable, such as by being devoid of slots 250, a distal section of steerable tube 210, extending immediately proximal to the steerable tube distal portion 214, is the section of the tube that includes the slotted section 246 or at least a portion of the slotted section 246, such that at least the distal section of steerable tube 210 is necessarily steerable. While a slotted section 246 is shown to extend only over a distal section of the steerable tube 210 in the illustrated example, it is to be understood that in some examples, slotted section 246 can extend further towards and optionally including the steerable tube proximal portion 228.

While the shape and dimensions of all slots 250 is shown to be identical in the illustrated example, it is to be understood that in some examples, at least some successive circumferential slots 250 can be differently shaped and/or differently dimensioned from each other. In some examples, at least some successive slots 250 can vary in length. In some examples, at least some successive slots 250 can vary in width, defined as the dimension of the slots 250 in a direction perpendicular to the their length.

Ribs 258 are defined as the portions of the slotted section 246 axially extending between adjacent slots 250. Each rib 258 circumferentially extends from a rib free end 260 defined at the corresponding longitudinal slot 230, to a rib backbone end 262. The width of a rib 258 is defines as the axial distance between circumferential slots 250 on both sides of the rib 258. While all ribs 258 are shown in the illustrated example to have similar widths, indicative of a uniform pitch of the slotted section 246, it is to be understood that in some examples, the width of at least some successive ribs 258 can vary, indicative of a varying pitch of the slotted section.

While circumferential slots 250 are defined over the slotted section 246 of the second tube portion 244, the first tube portion 242 remains devoid of circumferential slots 250. The first tube portion 242 and the second tube portion 244 are joined together at the steerable tube distal portion 214, which is a distal portion of the steerable tube 210 which is distal to the slotted section 246, and is devoid of both circumferential slots 250 and longitudinal slots 230. Thus, steerable tube distal portion 212 extends between the longitudinal slot distal ends 240 and a steerable tube distal edge 216.

The first tube portion 242 can be also referred to as a pullable portion of steerable tube 210, while the second tube portion 244 can be referred to as a non-pullable or stationary portion of the steerable tube 210. The first tube portion 242 can be coupled, for example inside the handle 204, to a mechanism (not shown) configured to apply an axial pull force thereto, and to release the pull force. The first tube portion 242 can be actuated by being proximally pulled, relative to the second tube portion 244, which effects bending of the slotted section 246.

FIG. 6A is a cross-sectional view of an exemplary steerable tube 210 in a first state, which can be also referred to as an unbent state, wherein the first portion proximal end 243 can be axially aligned with the second portion proximal end 245. The first tube portion 242 can be attached, optionally at the first portion proximal end 243, to an actuation mechanism configured to pull the first tube portion 242, thereby bending the slotted section 246 of steerable tube 210 as illustrated in FIG. 6B, optionally translating the first portion proximal end 243 proximal to the second portion proximal end 245. The steerable tube 210 can revert from the second state, which can be also referred to as the bent state shown in FIG. 6B, back to the first or unbent state of FIG. 6A, by releasing the pull force from the first tube portion 242.

In some examples, each longitudinal slot 230 comprises a longitudinal slot distal portion 232 extending proximally from the longitudinal slot distal end 240, a longitudinal slot proximal portion 236 extending distally from the longitudinal slot proximal end 238, and a longitudinal slot transitioning portion 234 extending between the longitudinal slot distal portion 232 and the longitudinal slot proximal portion 236. In some examples, the longitudinal slot distal portions 232 and longitudinal slot proximal portions 236 can be substantially parallel to the tube central axis C_{T}, but differently spaced from each other around the circumference of the steerable tube 210, such that the longitudinal slot distal portions 232 are circumferentially closer to each other than the longitudinal slot proximal portions 236. This means that the width of the first tube portion 242, in such examples, defined as the circumferential distance between both longitudinal slots 230, is greater along the portion defined between the longitudinal slot proximal portions 236 than between the longitudinal slot distal portions 232.

The width of the first tube portion 242 along the slotted section 246 can be dictated by the length of the circumferential slots 250. Longer slots 250 can optionally provide for greater flexibility of the slotted section 246. In contrast, the width of the first tube portion 242 along a proximal portion of the steerable tube 210 that no longer includes circumferential slots 250 does not have to be limited in a similar manner, and can be thus, in some examples, designed to be relatively wider, which can provide for a greater surface area for attachment of the proximal end portion of the first tube portion 242, such as along first portion proximal end 243, to a pulling mechanism within the handle 204.

Various exemplary implementations for delivery apparatuses 202 and/or steerable tubes 210 thereof can be referred to, throughout the specification, with superscripts, for ease of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, steerable tube 210^{a} is an exemplary implementation of steerable tube 210, and thus includes all of the features described for steerable tube 210 throughout the current disclosure, except that while a steerable tube 210 can be generally provided either as a steerable needle comprising a shaft tip, or as a steerable tube having a blunt distal opening, configured to accommodate a needle passable therethrough, steerable tube 210^{a} is implemented as a steerable needle.

FIG. 5A is a perspective view of a steerable tube implemented as a steerable needle 210^{a}, with the view angle directed towards the first tube portion 242. FIG. 5B is a perspective view of a distal region of the steerable needle 210^{a} of FIG. 5A, with the view angle directed towards the backbone 248. FIG. 5C is a side view of the steerable needle 210^{a} of FIGS. 5A-5B. FIGS. 6A and 6B show a steerable needle 210^{a} in a first and a second state, respectively. The term "steerable needle 210^{a}" refers to any steerable tube implemented to be in the form of a steerable needle. The distal portion 214^{a} of a steerable needle 210^{a} is configured to pierce a host leaflet 10 of a host valvular structure 12 to form a pilot puncture 50 in the host leaflet 10. The distal edge 216^{a} of steerable tube distal portion 214^{a} can define an angled surface 218. The angled surface 218 can terminate at a sharp piercing tip 219 to facilitate piercing the host leaflet 10 when the steerable needle 210^{a} is pressed against the leaflet.

FIGS. 7A and 7B show a sectional view in perspective and a side cross-sectional view of a distal region of an exemplary delivery apparatus 200. In some examples, a delivery apparatus 200 can include a dilator 270 that can be conical or frustoconical in shape, and include a dilator tapering portion 274 terminating at a dilator distal end 272, and a dilator proximal portion 276 that can be coupled to a dilator shaft 278 that extends proximally therefrom. A dilator lumen 280 continuously extends through the dilator shaft 278 and the dilator 270, open ended at the dilator distal end 272. Attachment of the dilator shaft 278 to the dilator proximal portion 276 can be achieved by a variety of methods, such as overmolding, radio-frequency welding, through an adhesive, and/or a combination thereof. In some examples (not illustrated), the dilator shaft 278 can extend through the entire length of the dilator 270, such that a distal end of the dilator shaft 278 is aligned with the dilator distal end 272. In some examples (not illustrated), the dilator shaft 278 is coupled to one or more components, such as collars or other connectors, which are in turn attached to the dilator 270.

In some examples, the delivery apparatus can further include a balloon catheter 282 having an inflatable balloon 298, which can be also referred to as a hole-dilating balloon. The balloon 298 is configured to transition between a radially deflated state and a radially inflated state. The balloon catheter 282 can define a balloon catheter lumen 286, through which the dilator shaft 278 can optionally extend. The balloon catheter 282 can extend through the handle 204 and be fluidly connectable to a fluid source (not shown) for inflating the balloon 298. The fluid source comprises an inflation fluid. The term "inflation fluid", as used herein, means a fluid (for example, saline, though other liquids or gas can be used) used for inflating the balloon 298. The inflation fluid source is in fluid communication with the balloon catheter lumen 286, such as by an annular space defined between the inner surface of balloon catheter 282 and the outer surface of a dilator shaft 278 disposed therein. This annular space is in fluid communication with one or more balloon catheter inflation openings 284 exposed to an internal cavity of the balloon 298 such that inflation fluid from the fluid source (for example, a syringe or a pump) can flow through the balloon catheter lumen 286 into balloon 298 to inflate it, for example during formation of leaflet opening 52. The pressure of the inflation fluid within balloon 298 may provide the force that allows it to dilate a leaflet opening 52. Further, the balloon catheter lumen 286 may be configured to withdraw fluid from the balloon 298 through the balloon catheter inflation opening(s) 284, to deflate the balloon 298.

The inflatable balloon 298 of delivery apparatus 200, utilized as a hole-dilating balloon, is different from a typical balloon used for expanding balloon-expandable prosthetic valves or stents, in that while a typical valve-expanding balloon is inflatable to a diameter that can allow expansion of a prosthetic valve to a functional diameter thereof, which can be similar to, or greater than (for example, in the case of valve over-expansion) the diameter of the native annulus in which the valve is deployed, the maximum diameter of a hole-dilating balloon 298 can be significantly smaller, configured to increase the size of a pilot puncture 50 to form a larger leaflet opening 52, optionally without tearing the host leaflet 10 (though in some examples, the host leaflet 10 may be still torn by a balloon 298). In some examples, the maximum diameter to which the hole-dilating balloon 298 can be inflated is equal to or less than 12 mm. In some examples, the maximum diameter to which the hole-dilating balloon 296 can be inflated is equal to or less than 10 mm.

In some examples, the balloon 298 is coupled to a distal end portion of the balloon catheter 282 at its proximal end, while the balloon's distal end can be coupled, directly or indirectly, to another component of the delivery apparatus 200, such as the dilator 270 or dilator shaft 278. In the illustrated example, the balloon 298 is shown to be coupled to the dilator proximal portion 276. The dilator proximal portion 276 can optionally include an outer step configured to accommodate the distal end of the balloon 298, such that the outer surface of the balloon 298 can be flush or otherwise relatively continuous with the outer surface of the dilator 270.

In some examples, the delivery apparatus 200 can further include a delivery shaft 288 defining a delivery shaft lumen 292 through which one or more other components of the delivery apparatus 200 can extend. The delivery shaft can be passed through the outer shaft lumen 207. In the illustrated example, the delivery shaft 288 is disposed around the balloon catheter 282 and balloon 298. In some examples, a delivery cone 290 is attached to a distal end of the delivery shaft 288, as illustrated. The delivery cone 290 can be conical or frustoconical in shape, and include a delivery cone tapering portion 296 terminating at a delivery cone distal end 294. The delivery shaft lumen 292 can continuously extend through delivery shaft 288 and delivery cone 290, being open ended at the delivery cone distal end 294. Attachment of the delivery shaft 288 to the delivery cone 290 can be achieved by a variety of methods, such as overmolding, radio-frequency welding, through an adhesive, and/or a combination thereof. In some examples (not illustrated), the delivery shaft 288 is coupled to one or more components, such as collars or other connectors, which are in turn attached to the delivery cone 290.

The outer shaft 208, the delivery shaft 288, the balloon catheter 282 and/or dilator shaft 278, and the steerable tube 210, can be configured to be axially movable relative to each other. For example, a proximally oriented movement of the delivery shaft 288 relative to the balloon catheter 282, or a distally oriented movement of the balloon catheter 282 relative to the delivery shaft 288, can expose the balloon 298 from the delivery shaft 288. Similarly, a proximally oriented movement of the dilator 270 relative to the steerable tube 210, or a distally oriented movement of the steerable tube 210 relative to the dilator 270, can expose the steerable tube distal portion 214. The dilator lumen 280 is sized to allow for axial movement of the steerable tube 210 therethrough.

In some examples, such as when the balloon 298 is attached at both ends thereof to the dilator 270 and balloon catheter 282, both the dilator 270 with dilator shaft 278 and the balloon catheter 282 can be configured to move simultaneously in the axial direction, without necessarily being axially movable relative to each other, or while axial movement of one relative to the other is limited. In such examples, the delivery apparatus 200 can be designed such that axial movement of one of the balloon catheter 282 causes the dilator shaft 278 to move therewith, or such that axial movement of one of the dilator shaft 278 causes the balloon catheter 282 to move therewith.

During delivery of the apparatus 200 towards the host valvular structure 12, the delivery cone distal end 294 can be position over a proximal end of the dilator tapering portion 274, such as proximate to the transition between the dilator tapering portion 274 and dilator proximal portion 276, such that the outer surfaces of the delivery cone tapering portion 296 and the dilator tapering portion 274 can together form a continuous tapering shape for easier passage through a patient's vasculature.

The proximal ends of the steerable tube 210, balloon catheter 282, delivery shaft 288, outer shaft 208, and/or dilator shaft 278, can be coupled to the handle 204. During delivery, the handle 204 can be maneuvered by an operator (for example, a clinician or a surgeon) to axially advance or retract components of the delivery apparatus 200, such as the steerable tube 210, balloon catheter 282, delivery shaft 288, outer shaft 208, and/or dilator shaft 278, through the patient's vasculature and/or along the target site of treatment, as well as to pull the first tube portion 242 to bend the steerable tube 210 or release the pull force to allow it to revert to the first state, and to inflate the balloon 298 mounted on the balloon catheter 282 so as to enlarge a leaflet opening 52, as will be elaborated in further detail below, and to deflate the balloon 298 and optionally retract the delivery apparatus 200.

FIGS. 7A-7B show an exemplary delivery apparatus 200^{a}, which is an exemplary implementation of delivery apparatus 200, and thus includes all of the features described for delivery apparatus 200 throughout the current disclosure, except that the steerable tube which is axially movable through the dilator lumen 280 of delivery apparatus 200^{a} is a steerable needle 210^{a} having a steerable tube distal portion 214^{a} equipped with a piercing tip 219.

FIGS. 8A-8H illustrate some steps in a method for utilizing a delivery apparatus 200 for forming an opening within a target tissue. An exemplary implementation of the method is illustrated in FIGS. 8A-8H with respect to forming a leaflet hole inside a host leaflet, which can be performed prior to implanting a guest prosthetic valve inside the host valvular structure. The delivery apparatus 200 can be used to perforate a host leaflet 10, such as a native leaflet 30 or a prosthetic valve leaflet 114 of a previously implanted prosthetic valve. While delivery apparatus 200^{a} comprising steerable needle 210^{a} is illustrated throughout FIGS. 8A-8H, it is to be understood that other examples of delivery apparatus 200 described in the current specification can be used in a similar manner with some steps modified according to the configuration of the apparatus, as will be further elaborated below.

The distal end portion of the delivery apparatus 200 is configured to be inserted into a patient’s vasculature, such as within an ascending aorta, and to be advanced towards the host leaflet 10. Positioning the delivery apparatus 200 relative to the host leaflet 10 may comprise advancing the delivery apparatus 200 toward the leaflet over a guidewire 80. The steerable tube lumen 212 of a steerable needle 210^{a} can be configured to accommodate a guidewire 80 that can extend through the steerable tube lumen 212. In such examples, the guidewire 80 can be inserted into the patient’s vasculature, and then the steerable needle 210^{a} and/or other shafts or tubes of the delivery apparatus 200 may be advanced toward the host leaflet 10 over the guidewire 80.

As mentioned above, a steerable tube 210, such as steerable needle 210^{a}, is configured to be selectively translated in the proximal or distal directions relative to another component of the delivery apparatus 200, such as dilator 270. In some examples, the dilator shaft 278 and the steerable tube 210 are configured to be movable axially relative to each other in the proximal and distal directions. The steerable tube 210 can be coupled to the handle 204, which can have one or more actuators (for example, in the form of rotatable knobs 206) that are operatively coupled to the steerable tube 210 to facilitate axial movement thereof.

During delivery, the steerable tube distal portion 214 of a steerable needle 210^{a} can be retained inside dilator lumen 280, such that the piercing tip 219 is at or proximal to dilator distal end 272 as illustrated in FIG. 8A. This position conceals the sharp edge of the steerable needle 210^{a} from the surrounding anatomy, to protect the anatomical structures from being engaged or punctured by the distal portion 214 of steerable needle 210^{a} during advancement towards the site of treatment.

In some examples, at least one catheter or shaft of the delivery apparatus 200, aside from steerable tube 210, can be steerable so as to navigate the distal portion of the delivery apparatus 200 toward the desired host leaflet 10, such as a leaflet that can be closer to the left coronary ostium. In some cases, orienting the delivery apparatus 200 sideways, towards a host leaflet 10, optionally in some proximity to the nadir of the leaflet, can orient the tube central axis C_{T} towards a host interior surface 14, which can be the interior surface of the aortic wall if the host valve is the native valve, or an interior surface of a frame of a previously implanted prosthetic valve serving as the host valve. In such cases, merely advancing a needle in the distal direction to expose it prior to penetrating through the host leaflet 10, can direct the needle towards the host interior surface 14 instead of the host leaflet 10 itself.

In the case of a steerable needle 210^{a}, the first tube portion 242 can be proximally pulled, as described above, to bend the slotted section 246 of the steerable needle 210^{a} in a similar manner to that described above with respect to FIG. 6B, advantageously orienting the steerable tube distal portion 214 toward the host leaflet 10, as illustrated in FIG. 8B, such that during advancement of the steerable needle 210^{a}, its distal portion 214 can contact and pierce through the host leaflet 10, without posing a risk of contacting and damaging adjacent anatomical structures, such as the host interior surface 14.

In some examples, the dilator 270 can be flexible enough to bend along with the steerable tube 210 as illustrated in FIG. 8B. In some examples, when the dilator 270 is disposed over a sufficient length of the slotted section 246 in a second state of the steerable tube 210, at least a portion of the dilator 270, such as the dilator tapering portion 274, may be passively bent therewith.

As shown in FIG. 8C, the steerable needle 210^{a} is configured to puncture the host leaflet 10 to form a pilot puncture 50 within host leaflet 10, for example when its distal portion 214 is axially translated relative to dilator 270. The guidewire 80 can be then advanced through the steerable tube lumen 212 to terminate distal to the pilot puncture 50 of host leaflet 10 as shown in FIG. 8D.

In some examples, a sleeve 266 is disposed around the steerable tube 210, configured to facilitate slidable axial movement of the steerable tube 210 the dilator 270 or any other component of the delivery apparatus through which the steerable tube 210 is configured to slide. The sleeve 266 can be coupled at its sleeve distal end 268 to a region of the steerable tube 210 distal to the slotted section 246, such as the steerable tube distal portion 214. FIGS. 6A-6B show an exemplary sleeve 266 that can be tightly disposed over at least part of the steerable tube 210, so as to cover at least the portion that includes the slotted section. The sleeve can be tightly disposed around the outer surface of the steerable tube 210 in the first state, as shown in FIG. 6A, and can be flexible enough to allow transitioning of the steerable tube 210 to the second state, as shown in FIG. 6B. In some examples, the sleeve 266 comprises a heat-shrink layer that can be formed of heat-shrink tubing or a heat-shrink tape wrapped around the steerable tube 210.

The sleeve 266 can comprise a lubricious or low-friction material, wherein a low coefficient of friction of the sleeve 266 can facilitate axial movement of the steerable tube 210 through the dilator lumen 280. This can be of advantage to allow for easier movement of the steerable tube 210 through the dilator lumen 280, for example toward host leaflet 10, particularly in the second state of the steerable tube 210 where the distance between portions of the ribs 258, such as closer to the rib backbone ends 262, can increase, which may cause interference engagement with a surrounding surface in the absence of the sleeve 266.

In some examples, when the steerable tube 210 is positioned inside of the pilot puncture, such that the steerable tube distal portion 214 is positioned distal to the host leaflet 10, and the sleeve 266 covering the steerable tube 210 also extends around the tube 210, past the pilot puncture 50, the sleeve 266 can be proximally pulled from around the tube 210 and out of the pilot puncture 50, such that the steerable tube 210 is no longer bound by the sleeve in a portion of the tube 210 extending through the host leaflet 10. This allows the first tube portion 242, in a second state of the steerable tube 210, to extend radially away from the second portion 244. The steerable tube 210 can be then pulled out of the host leaflet 10 in such a bent uncovered state, wherein the exposed tensioned second tube portion 244, passing through the host leaflet 50, can be configured to cut through the tissue in a manner that further expands the pilot puncture 50.

In some examples, the second tube portion 244 is narrow enough, in a circumferential direction defined between both longitudinal slots 230, such that it can function as a cutting wire thin enough to cut through the tissue as it axially passes therethrough. In some examples, the outer surface of the second tube portion is a rough surface, configured to frictionally cut through the tissue when axially moved therethrough or therealong. In some examples, the outer surface includes serrations or other cutting features, concealed when the steerable tube 210 is covered by the sleeve 266, but exposed to facilitate further cutting through the tissue when uncovered by the sleeve. The steerable tube 210 can be proximally moved in such a bent uncovered state to further cut through the host leaflet 10, such that the steerable tube is completely retracted from the host leaflet 10, or only partly pulled such that at the end of this step, a portion of the steerable tube 210 still extends past the pilot puncture 50, such that the steerable tube distal portion 214 is still positioned distal to the host leaflet 10. After cutting through the host leaflet 10 to expand the pilot puncture 50 as described above, the slotted section 246 can be re-covered by the sleeve 266.

Subsequent to forming the pilot puncture 50 and optionally advancing the guidewire 80, and as shown in FIG. 8E, the dilator 270 can be inserted into the pilot puncture 50 to expand the pilot puncture 50. As the dilator 270 is inserted into the host leaflet 10, the inherent resiliency of the leaflet 10 may urge the leaflet 10 radially inwardly against the dilator 270. While the dilator 270 may be flexible enough to bend along with the slotted section 246 of a steerable tube 210, it can still have sufficient stiffness to facilitate advancement thereof through the leaflet 10, wherein the gradually tapering shape of the dilator 270 facilitates expanding the pilot puncture 50 to a greater diameter.

In a subsequent step of the method, illustrated in FIG. 8F, the balloon 298 may be inserted within the pilot puncture 50, such as by further advancement of the dilator 270 with dilator shaft 278 and/or balloon catheter 282. The distal portion 214 of the steerable needle 210^{a} can be re-concealed within dilator lumen 280, such as due to advancement of dilator 270 in a distal direction over steerable tube distal portion 214, and/or retraction of steerable needle 210^{a} such that the piercing tip 219 is at or proximal to the dilator distal end 272, to avoid damage that may be caused to internal anatomical structures of the patient's body due to accidental contact with the piercing tip 219.

In some examples, the guidewire 80 can be advanced simultaneously with advancement of the steerable needle 210^{a} during formation of the pilot puncture 50. In some examples, the guidewire 80 can be advanced to terminate distal to the host leaflet 10 after formation of the pilot puncture 50 by the steerable needle 210^{a}. In some examples, the guidewire 80 can be advanced to terminate distal to the host leaflet 10 prior to advancement of the dilator 270 through the pilot puncture 50. In some examples, the guidewire 80 can be advanced simultaneously with advancement of the dilator 270 into and through the pilot puncture 50 after formation of the pilot puncture 50 by the steerable needle 210^{a}.

In some examples, the steerable needle 210^{a} can be retracted back into dilator lumen 280 prior to advancement of the dilator 270 into pilot puncture 50, in which case the dilator 270 can be guided through the pilot puncture 50 of host leaflet 10 over a guidewire 80 distally advanced into and through pilot puncture 50, optionally prior to retraction of the steerable needle 210^{a}.

With the balloon 298 received within the pilot puncture 50, inflating the balloon 298 to transition it from a radially deflated state (FIG. 8F) to a radially inflated state (FIG. 8G) can expand the pilot puncture 50 to form a leaflet opening 52 that is sized to receive the prosthetic valve 100 in the radially compressed or crimped configuration. After the balloon 298 is inflated to form the leaflet opening 52 as shown in FIG. 8G, the balloon 298 is deflated, as shown in FIG. 8H, optionally allowing for insertion of a guest prosthetic valve inside the leaflet opening 52.

In some examples, inflating the balloon 298 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In some examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the balloon 298 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

The delivery apparatus 200 may be configured to form the leaflet opening 52 in any of a variety of host valvular structures 12. In the example of FIGS. 8A-8H, the host valvular structure 12 can be the valvular structure 113 of a previously implanted prosthetic valve, such as the prosthetic valve 100a of FIG. 3. In such examples, using the delivery apparatus 200 as described herein to form the leaflet opening 52 in a previously implanted prosthetic valve may be followed by steps for implanting a guest prosthetic valve 100b within the previously implanted prosthetic valve 100a (for example, via a ViV procedure).

Similarly, the host valvular structure 12 in the example of FIGS. 8A-8H can be a valvular structure 29 of a native heart valve, such as the native aortic valve 20 shown in FIGS. 2A-2B. In such examples, the delivery apparatus 200 can be configured to puncture a native leaflet 30 of the native aortic valve 20. In some examples, the host valvular structure and/or the native valve may refer to another valve of a patient’s heart, such as a mitral valve, a pulmonary valve, or a tricuspid valve.

While illustrated and described above with respect to forming a leaflet opening 52 within a host leaflet 10, it is to be understood that the delivery apparatus 200 may be configured to form a tissue opening through other tissues in a patient's body. For example, prosthetic devices can be delivered to the left atrium or the left ventricle in a transseptal approach, wherein a delivery apparatus is passed through the vena cava, into the right atrium, and through the interatrial septum tissue. Such delivery approaches require puncturing the interatrial septum. Thus, in some examples, a delivery apparatus 200 may be utilized to form an opening through the interatrial septum, for example at the site of the fossa ovalis, which is a region of the septum containing tissue of lesser thickness than is typical of the rest of the septum. Thus, any example of delivery apparatus 200 described herein can be utilized in a manner similar to that described with respect to FIGS. 8A-8H or modifications thereof, to form a tissue opening, equivalent to leaflet opening 52 described with respect to FIGS. 8A-8H, in a target tissue, equivalent to a host leaflet 10 described with respect to FIGS. 8A-8H.

In some examples, some or all of the components of delivery apparatus 200 described herein can be part of a delivery assembly that includes a delivery apparatus carrying a prosthetic valve (examples not shown explicitly). Similarly, a delivery apparatus that includes a steerable tube according to any example of the current disclosure can be used for implantation of other prosthetic devices aside from prosthetic valves, such as stents or grafts.

A delivery apparatus equipped with a steerable tube can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

In some examples, after forming the leaflet opening 52, and optionally after deflating the balloon 298, a guest prosthetic valve 100 can be positioned in the valvular structure 12 in a compressed state thereof, and expanded therein to implant the guest prosthetic valve 100 inside the host valvular structure. In some examples, the guest prosthetic valve 100 can be positioned inside a leaflet opening 52 in a radially compressed state thereof, and expanded therein in a manner that modifies the host leaflet 10. Radially expanding the guest prosthetic valve 100 can be performed in any suitable manner, such as using any suitable valve expansion technique and/or mechanism that is known to the art. In some examples, radial expansion of the guest prosthetic valve 100 can be achieved by inflating an inflatable valve-expanding balloon on which the guest prosthetic valve is mounted. As mentioned above, in contrast to the hole-expanding balloon 298, the valve-expanding balloon (not shown) is configured to expand to a diameter which is significantly greater than a maximum diameter of the hole-expanding balloon 298.

In some examples, a delivery apparatus 200 is part of a delivery assembly that further includes the guest prosthetic valve 100 carried, in a radially compressed state thereof, over a component of the delivery apparatus. Exemplary delivery assemblies that include perforating members that can be implemented in the form of a needle, a first balloon that can be a hole-expanding balloon 298, and a second balloon that can be valve-expanding balloon, are described in U.S. Provisional Application Nos. 63/447,453 and 63/447,457, each of which is incorporated herein by reference in its entirety.

In some examples, a delivery apparatus 200 can be retracted from the host valvular structure 12 and the patient's body, optionally subsequent to deflation of balloon 298, while the guidewire 80 remains in position, extending through the leaflet opening 52. Positioning a guest prosthetic valve within the leaflet opening can be performed, in such examples, by advancing the guest prosthetic valve into the leaflet opening via over the same guidewire 80.

In some examples, more than one guidewire can be utilized in a method that includes forming the leaflet opening 52 by delivery apparatus 200 and positioning a guest prosthetic valve 100 therein. For example, a first guidewire 80 can be utilized in a method of forming a leaflet opening 52 by the delivery apparatus 200 following the steps described with respect to FIGS. 8A-8H herein, or modifications thereof, after which the delivery apparatus 200 can be retracted along with guidewire 80, and a separate guidewire can be then used for advancing a guest prosthetic valve in the host valvular structure. In some examples, a separate guidewire over which a guest prosthetic valve can be advanced, can extend alongside the guidewire 80 over which the delivery apparatus 200 extends.

In some examples, the guest prosthetic valve can be a mechanically-expandable prosthetic valve and radial expansion thereof can be achieved by actuating a mechanical actuator of the guest prosthetic valve to mechanically expand a frame of the guest prosthetic valve. In some examples, the guest prosthetic valve can be a self-expandable prosthetic valve that can be retained during delivery toward the host valvular structure in a capsule or other restraint disposed therearound, and valve expansion can be achieved by removing the capsule or other restraint from the guest prosthetic valve to allow it to radially self-expand within the host valvular structure.

With the guest prosthetic valve received within the leaflet opening 52, radial expansion thereof can serve to increase a size of the leaflet opening and/or to tear the leaflet. As a result, the valve's radial expansion can serve to modify the host leaflet 10 such that the leaflet does not obstruct a cell opening in a frame of the guest prosthetic valve or at least increases the exposed area of the host valvular structure and the guest prosthetic valve that is not covered or obstructed by the modified host leaflet 10 to permit access and sufficient perfusion to the adjacent coronary artery.

While methods disclosed herein can refer to forming a leaflet opening 52 in a host leaflet 10, prior to positioning and expanding a prosthetic valve 100, it is to be understood that any of the methods can comprise, in some examples, repeating one or more steps disclosed throughout the current specification to form a plurality of openings in the host valvular structure. For example, steps described above with respect to FIGS. 8A-8H or modifications thereof, as will be described herein below, can be performed for forming a first leaflet opening in a first host leaflet, after which the delivery apparatus 200 can be retracted from the first host leaflet and steered toward another host leaflet, after which the same steps can be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

For example, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In some examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. For example, the host valvular structure can be modified such that the guest prosthetic valve is implanted by being expanded in a leaflet opening of a first host leaflet that faces the left coronary artery, and such that the second leaflet opening is formed in a second host leaflet that faces the right coronary artery (or vice-versa).

In some examples, forming the first leaflet opening can be performed prior to forming the second leaflet opening. In some examples, forming the second leaflet opening can be performed prior to forming the first leaflet opening. In some examples, the order of forming leaflet openings is chosen such that the final leaflet opening is formed in the host leaflet in which a guest prosthetic valve 100 is to be positioned and expanded.

It is to be understood that the guest prosthetic valve 100 is not limited to being implanted within an opening 52 of a leaflet. For example, in cases where the delivery apparatus 200 is utilized to form a full tear in a host leaflet that extends to the coaptation edge of the leaflet, the guest prosthetic valve 100 can be positioned at a location between the leaflets of the host valvular structure 12 and then expanded. In such cases, the opening 52 may provide sufficient open space through which blood may flow into the coronary ostia, and/or through which additional access devices, such as coronary catheters, can pass during future interventional procedures.

As mentioned, any delivery apparatus and method of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 9 shows a previously implanted prosthetic valve 100a subsequent to forming the leaflet opening 52, for example subsequent to the method described above with respect to FIGS. 8A-8H. FIG. 10 shows a configuration in which a second prosthetic valve 100b has been expanded within the leaflet opening 52 of a host prosthetic valve 100a. In the example of FIG. 10, the guest prosthetic valve 100b is the same type of valve as the host prosthetic valve 100a. It is to be understood, however, that ViV procedures may be similarly applied to any other suitable valvular structures, such as different prosthetic valves and/or native heart valves. For example, the guest prosthetic valve 100b need not be the same type of valve as the host prosthetic valve 100a.

In the example of FIG. 9, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 10, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

FIGS. 11A and 11B show a view in perspective and a side view of an exemplary steerable tube 210^{b}. Steerable tube 210^{b} is an exemplary implementation of steerable tube 210, and thus includes all of the features described for steerable tube 210 throughout the current disclosure, except that unlike steerable needle 210^{a}, steerable tube 210^{b} is not formed as a needle, but rather has a blunt or atraumatic distal edge 216^{b} at its distal portion 214^{b}, devoid of a piercing tip or other sharp edges.

FIGS. 12A and 12B show a sectional view in perspective and a side cross-sectional view of a distal region of an exemplary delivery apparatus 200^{b}. Delivery apparatus 200^{b} is an exemplary implementation of delivery apparatus 200, and thus includes all of the features described for delivery apparatus 200 throughout the current disclosure, except that the delivery apparatus 200^{b} comprises the steerable tube 210^{b} and a separate perforating member 220 disposed inside and axially movable within the steerable tube lumen 212^{b}.

As shown in FIGS. 12A-12B, a steerable tube 210^{b} can be disposed inside, and axially movable within, a dilator lumen 280 in the same manner described above with respect to FIGS. 7A-7B. However, while a steerable tube 210^{a} of delivery apparatus 200^{a} has a steerable tube lumen 212 sized to allow passage of a guidewire 80 therethrough, the steerable tube 210^{b} of delivery apparatus 200^{b} has a steerable tube lumen 212 sized to allow passage of a perforating member 220 therethrough. A sleeve 266 can be disposed around the steerable tube 210^{b} to facilitate advancement thereof within the dilator lumen 280 as described above. Perforating member 220 defines a perforating member lumen 222, and comprises a perforating member distal end portion 224 configured to pierce a host leaflet 10 of a host valvular structure 12 to form a pilot puncture 50 in the host leaflet 10. In some examples, the perforating member distal end portion 224 of perforating member 220 is configured to be selectively translated in the proximal or distal directions relative to another component of the delivery apparatus 200, such as the steerable tube 210.

In some examples, the perforating member 220 may include and/or be a needle. As shown in the illustrated example, the distal end portion 224 of the perforating member 232 can define an angled surface 226 that terminates at a sharp piercing tip 227 to facilitate piercing the host leaflet 10 when the needle is pressed against the leaflet. The perforating member lumen 222 of perforating member 220 of delivery apparatus 200^{b} can be sized to allow passage of a guidewire 80 therethrough. In such examples, a guidewire 80 can be inserted into the patient’s vasculature, and then the perforating member 220 and/or other shafts of the delivery apparatus 200, including a steerable tube 210 disposed around the perforating member 220, may be advanced toward the host leaflet 10 over the guidewire 80.

In some examples, the guidewire 80 can be used as a perforating or lacerating member for forming a pilot puncture 50. In such examples, the guidewire 80 can be a relatively stiff wire having a distal tip 82 configured to pierce the host leaflet 10 when the guidewire 80 is pressed against the leaflet. In some examples, the guidewire 80 can include a radio-frequency (RF) energy delivery tip 82 to assist with penetration through the leaflet tissue. For this purpose, a suitable RF energy device may be coupled to the guidewire 80, and the RF energy device can apply the RF energy to the guidewire tip 82 to penetrate the host leaflet 10. In any examples disclosed herein wherein a guidewire is used to puncture a leaflet, the guidewire can be coupled to a source of RF energy that applies RF energy to the tip of the guidewire. When the guidewire 80 is used to pierce the leaflet 10, the perforating member 220 in the form of a needle can be omitted, or it can be used in combination with the guidewire 80 that forms an initial puncture in the leaflet 10. For example, the guidewire 80 can be used to form an initial pilot puncture 50, after which the perforating member 220 can be advanced through the leaflet to form a slightly larger pilot puncture for subsequent advancement of the dilator 270 through the host leaflet 10.

In some examples, the guidewire 80 is used as a perforating member without any additional separate perforating member, such as a needle, disposed thereover, such that the guidewire 80 can be utilized as the sole component that forms the pilot puncture 50, extending through steerable tube lumen 212 and being bendable in a desired orientation when the slotted section 246 is articulated.

In some examples, the guidewire 80 is used as a perforating member that can be used in addition to perforating member (for example, needle) 220, such that the guidewire 238 can form an initial puncture via a sharp tip 82 or an RF energy delivery tip 82, followed by penetration of the perforating member 220 into the leaflet 10 to form the pilot puncture 50, or a pilot puncture 50 which is greater in size than an initial puncture formed by the guidewire tip 82. In some examples, the guidewire 80 is used as a perforating member that can be similarly used in combination with a steerable tube implemented as a steerable needle 210^{a}.

In some examples, the guidewire tip 82 is not necessarily sharp enough or otherwise configured to puncture through the host leaflet 10, in which case the guidewire 80 can be utilized for advancement of the delivery apparatus 200 toward the valvular structure 12, but terminate in proximity of the host leaflet 10 without piercing through it, and the distal end portion 224 of perforating member 220, or distal end portion 214^{a} of steerable needle 210^{a}, can be then advanced toward and into the host leaflet 10, to form the pilot puncture 50 in a similar manner to that illustrated in FIG. 8C.

FIGS. 13A and 13B show a sectional view in perspective and a side cross-sectional view of a distal region of an exemplary delivery apparatus 200^{c}. Delivery apparatus 200^{c} is an exemplary implementation of delivery apparatus 200, and thus includes all of the features described for delivery apparatus 200 throughout the current disclosure, except that the delivery apparatus 200^{c} comprises a perforating member 220 disposed inside and axially movable within the dilator lumen 280, and a steerable tube 210^{b} is disposed inside, and is axially movable within, the perforating member lumen 222. Thus, the perforating member lumen 222 of perforating member 220 of delivery apparatus 200^{c} can be sized to allow passage of the steerable tube 210^{b} therethrough, while the steerable tube 210^{b} has a steerable tube lumen 212 sized to allow passage of a guidewire 80 therethrough. A sleeve 266 can be disposed around the steerable tube 210^{b} to facilitate advancement thereof within the needle lumen 222.

While a perforating member 220 is illustrated in FIGS. 12A-13B in the form of a hollow needle, it is to be understood that it can be implemented as any other type of perforating or lacerating members, including perforating members equipped with sharp edges along tips or edges thereof, perforating members that include blades, and/or lacerating members that include electrically conductive portions, such as RF energy delivery tips, edges, or other portions.

Utilization of delivery apparatus 200^{b} or 200^{c} can be similar to the method described above with respect to FIGS. 8A-8H, except that when the first tube portion 242 of steerable tube 210^{b} is pulled to bend the slotted section 246 of the steerable tube 210^{b}, it will similarly bend not only the portion of the dilator 270 disposed therearound, but also the portion of the perforating member 220 disposed therein in the case of delivery apparatus 200^{b}, or the perforating member 220 disposed therearound in the case of delivery apparatus 200^{c}, thus directing the perforating member 220 towards the host leaflet 10. The perforating member 220 of any of delivery apparatus 200^{b} or 200^{c} is then advanced to penetrate through the host leaflet 10 and form the pilot puncture 50 in a similar manner to that described for steerable needle 210^{a} with respect to FIG. 8C, wherein the steerable tube 210^{b} can be either advanced simultaneously with, or subsequent to, advancement of the perforating member 220 to form the pilot puncture 50, or it can remain situated proximal to host leaflet 10. The subsequent steps of the method can be generally similar to those described above with respect to FIGS. 8D-8H, mutatis mutandis.

While conventional steerable catheters include a series of circumferential slots, and a pull wire attached to a distal portion thereof, configured to bend the catheters when proximally pulled, utilization of a tube bendable by pulling a pull wire attached thereto may increase the cross-sectional profile due to the added thickness of the pull wire. A steerable tube 210 according to any example described above, can be formed from a tube cut (for example, laser-cut) to form the longitudinal slots 230 and circumferential slots 250. In contrast, a first tube portion 242 formed between the two longitudinal slots 230 fulfills the role of a pull wire, without increasing the steerable tube's profile since the pullable portion 242 is part of the tube itself.

While steerable tubes 210^{b} disclosed herein can be used in combination with a separate perforating member 220 that can be disposed inside their lumen 212 or around their outer surface, implementing the steerable tube to serve as a steerable needle 210^{a} has the advantage of combining both features of steerability and tissue perforation into a single tubular member, thus providing a smaller overall profile that can be passed through other components of a delivery apparatus 200, such as a dilator lumen 280, while minimizing the overall cross-sectional profile of the apparatus to allow for simpler passage thereof through narrower regions of a patient's vasculature.

While exemplary delivery apparatuses 200 disclosed herein, are illustrated to include a dilator 270, a hole-expanding balloon 298 mounted on a balloon catheter 282, as well as other components such as delivery shaft 288 and/or outer shaft 208, it is to be understood that this components are optional, and that in some examples, a delivery apparatus 200 can include a steerable tube 210, optionally implemented as a steerable needle 210^{a} or used in combination with a separate perforating member 220, while one or more of the other components can be separately advanced towards and through the pilot puncture 50 after retraction of the delivery apparatuses 200.

For example, a delivery apparatus 200 can be provided with a dilator 270 but not necessarily a balloon mounted on a balloon catheter. In such a case, the delivery apparatus 200 can be retracted from the host valvular structure 12 and the patient's body, such as subsequent to expanding the pilot puncture 50 by the dilator 270, while the guidewire 80 remains in position, extending through the pilot puncture 50. A hole-expanding balloon mounted on a balloon catheter can be then separately advanced, over the same guidewire 80, towards and into the pilot puncture 50, and similarly inflated therein, in a manner equivalent to that described above with respect to FIGS. 8F-8H, mutatis mutandis, to form the leaflet opening 52.

In some examples, a delivery apparatus 200 can be provided without a dilator. In such a case, the delivery apparatus 200 can be retracted from the host valvular structure 12 and the patient's body, such as subsequent to forming the pilot puncture 50, while the guidewire 80 remains in position, extending through the pilot puncture 50. A dilator attached to a dilator shaft, with or without a balloon mounted on a balloon catheter, can be then separately advanced over the same guidewire 80 towards and into the pilot puncture 50, and similarly passed therethrough, in a manner equivalent to that described above with respect to FIG. 8E, mutatis mutandis, to dilate the pilot puncture 50.

In some examples, more than one guidewire can be utilized in a method that includes forming the pilot puncture 50 by delivery apparatus 200 and expanding it to form a leaflet opening 52. For example, a first guidewire 80 can be utilized in a method of forming a pilot puncture 50 by the delivery apparatus 200 following the steps described with respect to FIGS. 8A-8D herein, or modifications thereof, mutatis mutandis, after which the delivery apparatus 200 can be retracted along with guidewire 80, and a separate guidewire can be then used for advancing a dilator and/or a balloon mounted on a balloon catheter towards the host valvular structure. In some examples, a separate guidewire over which a dilator and/or a balloon mounted on a balloon catheter can be advanced, can extend alongside the guidewire 80 over which the delivery apparatus 200 extends.

While a valve-expanding balloon 298 is described above and illustrated for expanding a pilot puncture 50 to form a leaflet opening 52, it is to be understood that other types of expansion members can be used instead of a balloon 298 in any of the methods and/or apparatuses described herein. For example, U.S. Provisional Application Nos. 63/335,739, which is incorporated herein by reference in its entirety, describes an expandable frame that can be used instead of a valve-expanding balloon.

Any of the tools, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A delivery apparatus, comprising:

a handle;

a steerable tube extending distally from the handle and defining a steerable tube lumen, the steerable tube comprising:

two longitudinal slots circumferentially spaced from each other and defining a first tube portion therebetween and a second tube portion;

a slotted section comprising a plurality of pairs of circumferential slots axially spaced from each other along the second tube portion, wherein each circumferential slot of the plurality of pairs of circumferential slots extends between a slot free end at one of the two longitudinal slots and a slot backbone end; and

a backbone opposite to the first tube portion, the backbone defined between the slot backbone ends of the plurality of pairs of circumferential slots; and

a steerable tube distal portion extending from longitudinal slot distal ends of the two longitudinal slots to a steerable tube distal edge;

wherein axial movement of the first tube portion and/or the second tube portion relative to each other is configured to change the slotted section between a first state and a second state, such that the radius of curvature of the slotted section in the second state is different than its radius of curvature in the first state.

Example 2. The delivery apparatus of any example herein, particularly of example 1, wherein the slotted section is more bent in the second state than in the first state.

Example 3. The delivery apparatus of any example herein, particularly of example 1 or 2, wherein the steerable tube distal portion is devoid of circumferential slots.

Example 4. The delivery apparatus of any example herein, particularly of any one of examples 1 to 3, further comprising a strain relief regions at the slot backbone ends of at least some of the plurality of pairs of circumferential slots.

Example 5. The delivery apparatus of any example herein, particularly of any one of examples 1 to 4, wherein the axial movement between the first tube portion and the second tube portion comprises proximal movement of the first tube portion relative to the second tube portion, configured to change the slotted section from the first state to the second state.

Example 6. The delivery apparatus of any example herein, particularly of example 5, wherein the slotted section is configured to revert from the second state to the first state when the first tube portion is no longer proximally pulled.

Example 7. The delivery apparatus of any example herein, particularly of any one of examples 1 to 6, wherein each of the two longitudinal slots comprises a longitudinal slot distal portion extending proximally from the longitudinal slot distal end, a longitudinal slot transitioning portion extending proximally from the longitudinal slot distal portion, and a longitudinal slot proximal portion extending proximally from the longitudinal slot transitioning portion, wherein a width of the first tube portion defined between the two longitudinal slot distal portions is less than a width of the first tube portion defined between the two longitudinal slot proximal portions.

Example 8. The delivery apparatus of any example herein, particularly of any one of examples 1 to 7, further comprising a sleeve disposed around the steerable tube.

Example 9. The delivery apparatus of any example herein, particularly of example 8, wherein the sleeve is attached, at a sleeve distal end thereof, to the steerable tube.

Example 10. The delivery apparatus of any example herein, particularly of example 9, wherein the sleeve distal end is attached to the steerable tube distal portion.

Example 11. The delivery apparatus of any example herein, particularly of example 8, wherein the sleeve comprises a heat shrink layer.

Example 12. The delivery apparatus of any example herein, particularly of example 11, wherein the heat shrink layer comprises a heat-shrink tape wrapped around the steerable tube.

Example 13. The delivery apparatus of any example herein, particularly of any one of examples 1 to 12, further comprising a dilator attached to a dilator shaft extending proximally therefrom towards the handle, wherein the steerable tube is disposed inside a dilator lumen defined by the dilator and the dilator shaft.

Example 14. The delivery apparatus of any example herein, particularly of example 13, wherein the steerable tube is axially movable relative to the dilator.

Example 15. The delivery apparatus of any example herein, particularly of example 13 or 14, wherein the dilator comprises a dilator tapering portion.

Example 16. The delivery apparatus of any example herein, particularly of example 15, wherein the dilator further comprises a dilator proximal portion which is proximal to the dilator tapering portion.

Example 17. The delivery apparatus of any example herein, particularly of any one of examples 13 to 16, wherein at least a portion of the dilator is configured to bend when the slotted section is in the second state therein.

Example 18. The delivery apparatus of any example herein, particularly of any one of examples 13 to 17, further comprising:

a balloon catheter extending distally from the handle and defining a balloon catheter lumen; and

a balloon mounted on the balloon catheter and in fluid communication with the balloon catheter lumen,

wherein the balloon is configured to transition between a radially deflated state and a radially inflated state.

Example 19. The delivery apparatus of any example herein, particularly of example 18, wherein the dilator shaft extends through the balloon catheter lumen.

Example 20. The delivery apparatus of any example herein, particularly of example 19, wherein the balloon is attached on one end to the balloon catheter, and on an opposite end to the dilator.

Example 21. The delivery apparatus of any example herein, particularly of example 19, wherein the balloon is attached on one end to the balloon catheter, and on an opposite end to the dilator shaft.

Example 22. The delivery apparatus of any example herein, particularly of any one of examples 18 to 21, further comprising a delivery shaft extending distally from the handle and disposed around the balloon catheter.

Example 23. The delivery apparatus of any example herein, particularly of example 22, wherein the balloon catheter is axially movable relative to the delivery shaft.

Example 24. The delivery apparatus of any example herein, particularly of example 22 or 23, wherein the dilator is axially movable relative to the delivery shaft.

Example 25. The delivery apparatus of any example herein, particularly of any one of examples 22 to 24, further comprising a delivery cone attached to, and extending distally from, the delivery shaft.

Example 26. The delivery apparatus of any example herein, particularly of example 25, wherein the delivery cone comprises a delivery cone tapering portion.

Example 27. The delivery apparatus of any example herein, particularly of any one of examples 22 to 26, further comprising an outer shaft extending distally from the handle and disposed around the delivery shaft.

Example 28. The delivery apparatus of any example herein, particularly of example 27, wherein the delivery shaft is axially movable relative to the outer shaft.

Example 29. The delivery apparatus of any example herein, particularly of any one of examples 1 to 28, wherein the steerable tube is a steerable needle.

Example 30. The delivery apparatus of any example herein, particularly of any one of examples 1 to 29, wherein the steerable tube distal edge defines an angled surface.

Example 31. The delivery apparatus of any example herein, particularly of example 30, wherein the angled surface terminates at a piercing tip.

Example 32. The delivery apparatus of any example herein, particularly of any one of examples 29 to 31, wherein the steerable tube is configured to pierce a host leaflet of a host valvular structure to form a pilot puncture in the host leaflet.

Example 33. The delivery apparatus of any example herein, particularly of any one of examples 1 to 28, further comprising a perforating member axially movable relative to the steerable tube.

Example 34. The delivery apparatus of any example herein, particularly of example 33, wherein the perforating member is a needle comprising a distal end portion defining an angled surface that terminates with a piercing tip.

Example 35. The delivery apparatus of any example herein, particularly of example 33 or 34, wherein the perforating member defines a perforating member lumen.

Example 36. The delivery apparatus of any example herein, particularly of any one of examples 33 to 35, wherein the perforating member is disposed within the steerable tube lumen.

Example 37. The delivery apparatus of any example herein, particularly of any one of examples 33 to 35, wherein the perforating member is disposed around the steerable tube.

Example 38. The delivery apparatus of any example herein, particularly of any one of examples 33 to 37, wherein the perforating member is configured to pierce a host leaflet of a host valvular structure to form a pilot puncture in the host leaflet.

Example 39. The delivery apparatus of any example herein, particularly of any one of examples 18 to 28, wherein the balloon, when positioned within a pilot puncture formed inside a host leaflet of a host valvular structure, is configured to expand the pilot puncture to form a leaflet opening.

Example 40. A method comprising:

advancing a delivery apparatus comprising a steerable tube, over a guidewire, to a host valvular structure, the steerable tube comprising: two longitudinal slots defining a first tube portion and a second tube portion, a steerable tube distal portion extending distally from the longitudinal slots, and a slotted section which comprises a plurality of pairs of circumferential slots disposed along the second tube portion and defining a backbone opposite to the first tube portion;

bending the slotted section by axially moving the first tube portion and the second tube portion relative to each other; and

forming, with a piercing tip of the delivery apparatus, a pilot puncture within a host leaflet of the host valvular structure.

Example 41. The method of any example herein, particularly of example 40, wherein the bending the slotted section comprises orienting the piercing tip towards the host leaflet.

Example 42. The method of any example herein, particularly of example 40 or 41, wherein the forming the pilot puncture comprises distally advancing the piercing tip towards and through the host leaflet.

Example 43. The method of any example herein, particularly of any one of examples 40 to 42, wherein the steerable tube is a steerable needle, and wherein the steerable tube distal portion comprises the piercing tip.

Example 44. The method of any example herein, particularly of example 43, wherein the steerable tube distal portion comprises a steerable tube distal edge that defines an angled surface.

Example 45. The method of any example herein, particularly of example 43 or 44, wherein the steerable tube comprises a steerable tube lumen sized to allow passage of the guidewire therethrough.

Example 46. The method of any example herein, particularly of any one of examples 40 to 42, further comprising a perforating member which is axially movable relative to the steerable tube.

Example 47. The method of any example herein, particularly of example 46, wherein the perforating member is coaxial with the steerable tube.

Example 48. The method of any example herein, particularly of example 46 or 47, wherein the perforating member is a perforating member comprising a perforating member distal end portion which comprises the piercing tip.

Example 49. The method of any example herein, particularly of any one of examples 46 to 48, wherein the bending the slotted section of the steerable tube causes the perforating member to bend therewith.

Example 50. The method of any example herein, particularly of any one of examples 46 to 49, wherein the perforating member is disposed inside a steerable tube lumen of the steerable tube.

Example 51. The method of any example herein, particularly of example 50, wherein the perforating member comprises a perforating member lumen sized to allow passage of the guidewire therethrough.

Example 52. The method of any example herein, particularly of any one of examples 46 to 49, wherein the steerable tube is disposed inside a perforating member lumen of the perforating member.

Example 53. The method of any example herein, particularly of example 52, wherein the steerable tube comprises a steerable tube lumen sized to allow passage of the guidewire therethrough.

Example 54. The method of any example herein, particularly of any one of examples 40 to 53, further comprising a sleeve disposed around the steerable tube.

Example 55. The method of any example herein, particularly of example 54, wherein the sleeve is attached, at a sleeve distal end thereof, to the steerable tube.

Example 56. The method of any example herein, particularly of example 55, wherein the sleeve distal end is attached to the steerable tube distal portion.

Example 57. The method of any example herein, particularly of example 54, wherein the sleeve comprises a heat shrink layer.

Example 58. The method of any example herein, particularly of example 57, wherein the heat shrink layer comprises a heat-shrink tape wrapped around the steerable tube.

Example 59. The method of any example herein, particularly of any one of examples 40 to 58, wherein the steerable tube distal portion is devoid of circumferential slots.

Example 60. The method of any example herein, particularly of any one of examples 40 to 59, further comprising a strain relief regions at the slot backbone ends of at least some of the plurality of pairs of circumferential slots.

Example 61. The method of any example herein, particularly of any one of examples 40 to 60, wherein the axially moving comprises proximally pulling the first tube portion relative to the second tube portion.

Example 62. The method of any example herein, particularly of example 61, wherein the slotted section is configured to revert to an unbent state when the first tube portion is no longer proximally pulled.

Example 63. The method of any example herein, particularly of any one of examples 40 to 62, wherein each of the two longitudinal slots comprises a longitudinal slot distal portion extending proximally from the longitudinal slot distal end, a longitudinal slot transitioning portion extending proximally from the longitudinal slot distal portion, and a longitudinal slot proximal portion extending proximally from the longitudinal slot transitioning portion, wherein a width of the first tube portion defined between the two longitudinal slot distal portions is less than a width of the first tube portion defined between the two longitudinal slot proximal portions.

Example 64. The method of any example herein, particularly of any one of examples 40 to 63, further comprising a dilator defining a dilator lumen, wherein the steerable tube is disposed inside the dilator lumen.

Example 65. The method of any example herein, particularly of example 64, wherein the steerable tube and the dilator are axially movable relative to each other.

Example 66. The method of any example herein, particularly of example 64 or 65, further comprising a dilator shaft attached to, and extending proximally from, the dilator.

Example 67. The method of any example herein, particularly of any one of examples 64 to 66, wherein the dilator comprises a dilator tapering portion terminating at a dilator distal end.

Example 68. The method of any example herein, particularly of example 67, wherein the advancing a delivery apparatus comprises retaining the piercing tip proximal to the dilator distal end.

Example 69. The method of any example herein, particularly of example 67 or 68, wherein the forming the pilot puncture comprises advancing the piercing tip distally from the dilator distal end, while maintaining the dilator distal end proximal to the host leaflet.

Example 70. The method of any example herein, particularly of any one of examples 64 to 69, further comprising, subsequent to the forming the pilot puncture, passing the dilator through the pilot puncture, thereby expanding the pilot puncture.

Example 71. The method of any example herein, particularly of any one of examples 64 to 70, further comprising, subsequent to the forming the pilot puncture, extending the guidewire to terminate distally to the pilot puncture of the host leaflet.

Example 72. The method of any example herein, particularly of any one of examples 64 to 71, further comprising, subsequent to the forming the pilot puncture, concealing the piercing tip inside the dilator lumen.

Example 73. The method of any example herein, particularly of any one of examples 64 to 72, wherein the bending the slotted section of the steerable tube causes at least a portion of the dilator to bend therewith.

Example 74. The method of any example herein, particularly of example 70, further comprising a balloon catheter defining a balloon catheter lumen, and a balloon mounted on the balloon catheter and in fluid communication with the balloon catheter lumen.

Example 75. The method of any example herein, particularly of example 74, wherein the balloon is attached on one end to the balloon catheter, and on an opposite end to the dilator.

Example 76. The method of any example herein, particularly of example 74 or 75, further comprising, subsequent to the passing the dilator through the pilot puncture, positioning the balloon, in a radially deflated state thereof, within the pilot puncture.

Example 77. The method of any example herein, particularly of example 76, further comprising, subsequent to the positioning the balloon, inflating the balloon to expand the pilot puncture and form a leaflet opening within the host leaflet.

Example 78. The method of any example herein, particularly of example 77, further comprising, subsequent to the inflating the balloon, deflating the balloon.

Example 79. The method of any example herein, particularly of any one of examples 74 to 78, further comprising a delivery shaft disposed around the balloon catheter.

Example 80. The method of any example herein, particularly of example 79, wherein the balloon catheter is axially movable relative to the delivery shaft.

Example 81. The method of any example herein, particularly of example 79 or 80, wherein the dilator is axially movable relative to the delivery shaft.

Example 82. The method of any example herein, particularly of any one of examples 79 to 81, further comprising a delivery cone attached to, and extending distally from, the delivery shaft.

Example 83. The method of any example herein, particularly of example 82, wherein the delivery cone comprises a delivery cone tapering portion terminating at a delivery cone distal end.

Example 84. The method of any example herein, particularly of example 83, wherein the advancing a delivery apparatus comprises maintaining the delivery cone distal end around the dilator.

Example 85. The method of any example herein, particularly of example 83 or 84, wherein the passing the dilator through the pilot puncture comprises maintaining the delivery cone distal end proximal to the host leaflet.

Example 86. The method of any example herein, particularly of any one of examples 82 to 85, further comprising an outer shaft disposed around the delivery shaft.

Example 87. The method of any example herein, particularly of example 86, wherein the delivery shaft is axially movable relative to the outer shaft.

Example 88. The method of any example herein, particularly of example 77 or 78, further comprising, subsequent to the forming the leaflet opening, positioning a guest prosthetic valve in a radially compressed state thereof within the host valvular structure, and radially expanding the guest prosthetic valve.

Example 89. The method of any example herein, particularly of example 88, wherein the positioning the guest prosthetic valve within the host valvular structure comprises positioning the guest prosthetic valve within the leaflet opening.

Example 90. The method of any example herein, particularly of example 88 or 89, wherein the radially expanding the guest prosthetic valve comprises inflating a valve-expanding balloon over which the guest prosthetic valve is disposed.

Example 91. The method of any example herein, particularly of example 88 or 89, wherein the radially expanding the guest prosthetic valve comprises actuating a mechanical actuator of the guest prosthetic valve.

Example 92. The method of any example herein, particularly of example 88 or 89, wherein the guest prosthetic valve is a self-expandable prosthetic valve, and wherein radially expanding the guest prosthetic valve comprises removing a restraint from around the guest prosthetic valve.

Example 93. The method of any example herein, particularly of any one of examples 40 to 92, wherein the host valvular structure is a native valvular structure of native heart valve.

Example 94. The method of any example herein, particularly of any one of examples 40 to 92, wherein the host valvular structure is a valvular structure of previously implanted prosthetic valve that is implanted within a native heart valve.

Example 95. A leaflet piercing device comprising:

a tube defining a lumen extending along a longitudinal axis, the tube comprising:

a first longitudinal slot;

a second longitudinal slot;

a first tube portion longitudinally extending between the first and second longitudinal slots;

a second tube portion opposite of the first tube portion and comprising a backbone;

a plurality of circumferential slots circumferentially extending between the first and second longitudinal slots and the backbone; and

a piercing distal tip,

wherein a relative longitudinal movement of the first tube portion relative to the second tube portion is configured to bend the tube such that the piercing distal tip is offset from the longitudinal axis.

Example 96. A method of forming a puncture in a host leaflet, the method comprising:

advancing a tube, over a guidewire, to a host valvular structure, the tube comprising:

a slotted section comprising a first longitudinal slot, a second longitudinal slot, a backbone, and a plurality of circumferential slots formed between a first longitudinal slot and a second longitudinal slot;

a first tube portion extending between the first longitudinal slot and the second longitudinal slot;

a second tube portion; and

a distal piercing tip;

moving the first tube portion relative to the second tube portion to change the slotted section between a first configuration and a second configuration; and

advancing the tube to form a puncture within a host leaflet with the distal piercing tip.

The following examples are described in and based on International Patent Application Publications WO 2025/064478A1.

Example 1a. A steerable delivery system comprising: a handle; and a steerable catheter assembly extending distally from the handle and comprising: a steerable assembly lumen defining a steerable assembly central longitudinal axis; an inner tube comprising: an inner tube slotted portion defining an inner tube outer surface oriented away from the steerable assembly central longitudinal axis; and an inner tube distal end portion distal to the inner tube slotted portion; an outer tube disposed around the inner tube, the outer tube comprising: an outer tube slotted portion defining an outer tube inner surface oriented towards the steerable assembly central longitudinal axis; and an outer tube distal end portion distal to the inner tube slotted portion; and a pull-member comprising: a pull-ring portion affixed to the inner tube distal end portion and to the outer tube distal end portion; and at least one elongated pull-arm extending proximally from the pull-ring portion, wherein the at least one elongated pull-arm is disposed between the inner tube outer surface and the outer tube inner surface, and is axially slidable relative to the inner tube slotted portion and the outer tube slotted portion; wherein the at least one elongated pull-arm defines a circumferential width between arm circumferential ends that is greater than a radial thickness of the at least one elongated pull-arm; and wherein the at least one elongated pull-arm is configured to bend the steerable catheter assembly when the at least one elongated pull-arm is proximally pulled.

Example 2a. The system of Example 1a, wherein the at least one elongated pull-arm is integrally formed with the pull-ring portion.

Example 3a. The system of any one of Examples 1a to 2a, wherein the at least one elongated pullarm comprises a plurality of elongated pull-arms.

Example 4a. The system of Example 3a, wherein the plurality of elongated pull-arms comprises four elongated pull-arms disposed at 90° from each other.

Example 5a. The system of any one of Example 1a to 4a, wherein the circumferential width of the at least one elongated pull-arm is at least two times as great as its radial thickness.

Example 6a. The system of any one of Example 1a to 5a, wherein the at least one elongated pullarm comprises a curved arm inner surface oriented towards the steerable assembly central longitudinal axis, and a curved arm outer surface oriented away from the steerable assembly central longitudinal axis.

Example 7a. The system of any one of Example 1a to 6a, wherein the at least one elongated pullarm comprises an arm proximal portion extending into the handle, and wherein the circumferential width of the elongated pull-arm at the arm proximal portion is greater than the circumferential width of the elongated pull-arm at a portion of the at least one elongated pullarm disposed between the inner tube slotted portion and the outer tube slotted portion.

Example 8a. The system of Example 7a, further comprising a steering plate disposed inside the handle, wherein the arm proximal portion is attached to the steering plate, and wherein the steering plate is configured to tilt relative to the steerable assembly central longitudinal axis.

Example 9a. The system of any one of Example 1a to 8a, further comprising a delivery catheter extending distally from the handle, the delivery catheter comprising a primary lumen through which the steerable catheter assembly extends.

Example 10a. The system of Example 9a, wherein the delivery catheter is a steerable delivery catheter comprising a slotted tube disposed around the primary lumen, and a pull-wire lumen extending along at least a portion of the steerable delivery catheter, and wherein the system further comprises a pull-wire slidingly extending through the pull-wire lumen and attached to the slotted tube.

Example 11a. The system of Example 10a, wherein the pull- wire defines a pull-wire diameter that is greater than the radial thickness of the at least one elongated pull-arm.

Example 12a. The system of any one of Example 1a to 11a, further comprising a perforating member defining a perforating member lumen, the perforating member extending through the steerable assembly lumen.

Example 13a. The system of Example 12a, wherein the perforating member is configured to pierce a target tissue to form a pilot puncture in the target tissue.

Example 14a. The system of Example 13a, further comprising an expansion member configured to expand a pilot puncture formed in a target tissue by the perforating member.

Example 15a. A method comprising : advancing a steerable delivery system comprising a steerable catheter assembly, over a guidewire, to a target tissue, the steerable catheter assembly comprising an inner tube, an outer tube disposed around the inner tube, and a pull-member that comprises a pull-ring portion affixed to an inner tube distal end portion of the inner tube and to an outer tube distal end portion of the outer tube, and at least one elongated pull-arm extending proximally from the pull-ring portion and which is slidingly movable between and relative to the inner tube and the outer tube; bending a distal portion of the steerable catheter assembly by proximally pulling the at least one elongated pull-arm; and forming, with a perforating member tip of a perforating member extending through a steerable assembly lumen defined by the steerable catheter assembly, a pilot puncture within a target tissue; and wherein the at least one elongated pull-arm defines a circumferential width between arm circumferential ends that is greater than a radial thickness of the at least one elongated pull.

Example 16a. The method of Example 15a, wherein the pulling the at least one elongated pull- arm comprises tilting a steering plate to which an arm proximal portion of the at least one elongated pull- arm is attached.

Example 17a. The method of any one of Examples 1a to 16a, wherein the steerable delivery system further comprises a delivery catheter defining a primary lumen through which the steerable catheter assembly extends, and a slotted tube disposed around the primary lumen, and wherein the method further comprises, prior to the bending the distal portion of the steerable catheter assembly, bending the steerable delivery catheter.

Example 18a. The method of Example 17a, wherein the bending the distal portion of the steerable catheter assembly comprises bending the distal portion of the steerable catheter assembly at a different bending direction than a bending direction of the steerable delivery catheter.

Example 19a. The method of Example 17a or 18a, wherein the bending the steerable delivery catheter comprises proximally pulling a pull-wire attached to the slotted tube.

Example 20a. The method of any one of Example 15a to 19a, wherein the steerable delivery system further comprises an expansion member axially movable relative to the steerable catheter assembly.

Example 21a. The method of Example 20a, further comprising, after the forming the pilot puncture, positioning the expansion member inside the pilot puncture, in a compacted state of the expansion member.

Example 22a. The method of Example 21a, further comprising, subsequent to the positioning the expansion member inside the pilot puncture, expanding the expansion member to expand the pilot puncture and form a tissue opening within the target tissue.

Example 23a. A steerable delivery assembly comprising: an inner tube comprising an inner tube slotted portion and an inner tube distal portion distal to the inner tube slotted portion; an outer tube comprising an outer tube slotted portion and an outer tube distal portion distal to the outer tube slotted portion; a pull-member comprising: a distal portion affixed to the inner tube distal end portion or the outer tube distal end portion; and at least one elongated pull-arm extending proximally from the distal portion.

Example 24a. The steerable delivery assembly of Example 23a, wherein the at least one elongated pull-arm defines a circumferential width between arm circumferential ends that is greater than a radial thickness of the at least one elongated pull-arm.

Example 25a. The steerable delivery assembly of any one of Example 23a to 24a, wherein the at least one elongated pull-arm is configured to bend the steerable catheter assembly when the at least one elongated pull-arm is proximally pulled.

Example 26a. The steerable delivery assembly of any one of Example 23a to 25a, wherein the distal portion of the pull-member is a ring positioned between the inner tube distal end portion or the outer tube distal end portion.

Example 27a. The steerable delivery assembly of any one of Example 23a to 26a, wherein the at least one elongated pull-arm is disposed between the inner tube outer surface and the outer tube inner surface, and is axially slidable relative to the inner tube slotted portion and the outer tube slotted portion.

The following examples are described in and based on International Patent Application Publications WO2025072244A1.

Example 1b. A stabilized tissue modification system, comprising: an anchor device comprising: an anchor shaft defining an anchor shaft lumen; a helical anchor head coupled to the anchor shaft, the helical anchor head defining an anchor channel in fluid communication with the anchor shaft lumen, the helical anchor head comprising: an anchor proximal end; and at least one helical slot extending distally from the anchor proximal end, the at least one helical slot defining one or more helical turns, wherein a distal- most helical turn of the one or more helical turns defines a tip portion terminating at an anchor tip; a balloon catheter defining a balloon catheter lumen; and a hole-dilating balloon mounted on the balloon catheter and in fluid communication with the balloon catheter lumen, the hole-dilating balloon configured to transition between deflated and inflated states thereof; wherein the anchor shaft is a flexible torque shaft configured to rotate around a central axis thereof, such that when the anchor shaft is rotated, the helical anchor head is configured to rotate therewith.

Example 2b. The system of Example 1b, wherein the anchor shaft is configured to be axially advanced over a guidewire, and wherein the balloon catheter is configured to be axially advanced over the same guide wire.

Example 3b. The system of any one of Example 1b or 2b, wherein the helical slot has a width that tapers towards a slot proximal end thereof.

Example 4b. The system of any one of Example 1b to 3b, wherein the helical anchor head comprises a thinned wall portion terminating at a slot proximal end of the helical slot.

Example 5b. The system of any one of Example 1b to 4b, wherein the tip portion further comprises a cutting edge opposite to an inner side of the tip portion.

Example 6b. The system of Example 5b, wherein a length of the cutting edge spans at least 90° from the anchor tip.

Example 7b. The system of any one of Example 1b to 6b, wherein a thickening length of the tip portion spans at least 90° from the anchor tip.

Example 8b. The system of any one of Example 1b to 7b, wherein the helical anchor head is configured to form a pilot opening in a target tissue by gradually increasing a circumferential length of a cut formed in the target tissue during rotational movement of the helical anchor head through the target tissue.

Example 9b. The system of Example 8b, wherein inflation of the hole-dilating balloon, while positioned inside the pilot opening, is configured to expand the pilot opening to form a leaflet opening.

Example 10b. The system of any one of Example 1b to 9b, further comprising a needle defining a needle lumen, the needle extending through the anchor shaft lumen.

Example 11b. The system of Example 10b, wherein the needle is axially movable relative to the anchor shaft, wherein the anchor shaft further comprises a movement limiting segment defining a limiting segment channel which is continuous with the anchor shaft lumen, and wherein the needle comprises a stopper extending radially outward therefrom, the stopper configured to axially translate within the limiting segment channel.

Example 12b. A method comprising: advancing an anchor device of a stabilized tissue modification system, over a guidewire, to a host valvular structure, wherein the anchor device comprises an anchor shaft and a helical anchor head distal to the anchor shaft; and securing the helical anchor head to a host leaflet of the host valvular structure.

Example 13b. The method of Example 12b, further comprising: forming, with a perforating component of the stabilized tissue modification system, a pilot opening in the host leaflet; passing the guidewire through the pilot opening; releasing the helical anchor head from the host leaflet; positioning a hole-dilating balloon, mounted on a balloon catheter of the stabilized tissue modification system, inside the pilot opening, in a radially deflated state of the holedilating balloon; and inflating the hole-dilating balloon to expand the pilot opening and form a leaflet opening within the host leaflet.

Example 14b. The method of Example 13b, wherein the perforating component comprises a cutting edge extending along a tip portion of a distal-most helical turn of the helical anchor head.

Example 15b. The method of any one of Example 13b or 14b, wherein the forming the pilot opening comprises forming a cut, through the host leaflet, which gradually increases in length in the circumferential direction, during rotational movement of the helical anchor head, and wherein the formed cut spans less than 360° around a central axis of the helical anchor head.

Example 16b. The method of any one of Example 13b to 15b, wherein the stabilized tissue modification system further comprises a needle having a needle head, wherein the perforating component is a sharp needle tip of the needle head, and wherein the forming the pilot opening comprises perforating the host leaflet by the needle head.

Example 17b. The method of any one of Example 13b to 16b, further comprising, subsequent to the forming the pilot opening and prior to the positioning the hole-dilating balloon inside the pilot opening, passing a dilator through the pilot opening, thereby expanding the pilot opening.

Example 18b. The method of any one of Example 13b to 17b, further comprising, subsequent to the forming the leaflet opening, positioning a guest prosthetic valve in a radially compressed state thereof within the host valvular structure, and radially expanding the guest prosthetic valve.

Example 19b. The method of any one of Example 13b to 18b, further comprising, before the advancing the anchor device, advancing an outer shaft of a steerable delivery apparatus of the stabilized tissue modification system towards the host valvular structure, wherein the outer shaft defines an outer shaft lumen and distally extends from a handle of the steerable delivery apparatus, and wherein the handle of the steerable delivery apparatus comprises a rear port.

Example 20b. The method of any one of Example 13b to 19b, wherein the inflating the hole-dilating to expand the pilot opening and form the leaflet opening within the host leaflet comprises inflating a first hole-dilating balloon, mounted on a first balloon catheter, to expand a first pilot opening and form a first leaflet opening in a first host leaflet, and wherein the method further comprises: deflating and retrieving the first hole-dilating balloon; advancing the anchor device to the host valvular structure; securing the helical anchor head to a second host leaflet of the host valvular structure; forming, with the perforating component, a second pilot opening in the second host leaflet; releasing the helical anchor head from the second host leaflet; positioning a second hole-dilating balloon, mounted on a second balloon catheter of the stabilized tissue modification system, inside the second pilot opening, in a radially deflated state of the second hole-dilating balloon; and inflating the second hole-dilating balloon to expand the second pilot opening and form a second leaflet opening within the second host leaflet.

Example 21b. The method of Example 20b, wherein the first hole-dilating balloon is configured to expand to a maximum diameter which is greater than a maximum diameter to which the second hole-dilating balloon is configured to expand.

Example 22b. A stabilized tissue modification system, comprising: an anchor shaft comprising a helical anchor head, the helical anchor head comprising at least one helical slot defining one or more helical turns: a balloon catheter; a needle; and a hole-dilating balloon mounted on the balloon catheter, the hole-dilating balloon configured to transition between deflated and inflated states thereof.

Example 23b. The tissue modification system of Example 22b, wherein the anchor shaft is a flexible torque shaft configured to rotate around a central axis thereof, such that when the anchor shaft is rotated, the helical anchor head is configured to rotate therewith.

Example 24b. The tissue modification system of Example 22b or claim 23b, wherein a distal-most helical turn of the one or more helical turns defines a tip portion terminating at an anchor tip.

Example 25b. The tissue modification system of any one of Example 22b to 24b, wherein the helical anchor head is configured to form a pilot opening in a target tissue during rotational movement of the helical anchor head through the target tissue.

Example 26b. The tissue modification system of Example 25b, wherein inflation of the holedilating balloon, while the hold-dilating balloon is positioned inside the pilot opening, is configured to expand the pilot opening to form a leaflet opening.

Example 27b. The tissue modification system of any one of Example 22b to 26b, wherein the needle extends through the anchor shaft, wherein the needle is axially movable relative to the anchor shaft towards a target tissue to form a pilot opening.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A delivery apparatus, comprising:
a handle;
a steerable tube extending distally from the handle and defining a steerable tube lumen, the steerable tube comprising:
two longitudinal slots circumferentially spaced from each other and defining a first tube portion therebetween and a second tube portion;
a slotted section comprising a plurality of pairs of circumferential slots axially spaced from each other along the second tube portion, wherein each circumferential slot of the plurality of pairs of circumferential slots extends between a slot free end at one of the two longitudinal slots and a slot backbone end; and
a backbone opposite to the first tube portion, the backbone defined between the slot backbone ends of the plurality of pairs of circumferential slots; and
a steerable tube distal portion extending from longitudinal slot distal ends of the two longitudinal slots to a steerable tube distal edge;
wherein axial movement of the first tube portion and/or the second tube portion relative to each other is configured to change the slotted section between a first state and a second state, such that the radius of curvature of the slotted section in the second state is different than its radius of curvature in the first state.

2. The delivery apparatus of claim 1, wherein the slotted section is more bent in the second state than in the first state.

3. The delivery apparatus of claim 1, wherein the axial movement between the first tube portion and the second tube portion comprises proximal movement of the first tube portion relative to the second tube portion, configured to change the slotted section from the first state to the second state.

4. The delivery apparatus of claim 3, wherein the slotted section is configured to revert from the second state to the first state when the first tube portion is no longer proximally pulled.

5. The delivery apparatus of claim 1, wherein each of the two longitudinal slots comprises a longitudinal slot distal portion extending proximally from the longitudinal slot distal end, a longitudinal slot transitioning portion extending proximally from the longitudinal slot distal portion, and a longitudinal slot proximal portion extending proximally from the longitudinal slot transitioning portion, wherein a width of the first tube portion defined between the two longitudinal slot distal portions is less than a width of the first tube portion defined between the two longitudinal slot proximal portions.

6. The delivery apparatus of claim 1, further comprising a sleeve disposed around the steerable tube.

7. The delivery apparatus of claim 6, wherein the sleeve is attached, at a sleeve distal end thereof, to the steerable tube.

8. The delivery apparatus of claim 6, wherein the sleeve comprises a heat shrink layer.

9. The delivery apparatus of claim 1, further comprising a dilator attached to a dilator shaft extending proximally therefrom towards the handle, wherein the steerable tube is disposed inside a dilator lumen defined by the dilator and the dilator shaft.

10. The delivery apparatus of claim 9, further comprising:
a balloon catheter extending distally from the handle and defining a balloon catheter lumen; and
a balloon mounted on the balloon catheter and in fluid communication with the balloon catheter lumen,
wherein the balloon is configured to transition between a radially deflated state and a radially inflated state.

11. The delivery apparatus of claim 1, wherein the steerable tube is a steerable needle.

12. The delivery apparatus of claim 11, wherein the steerable tube is configured to pierce a host leaflet of a host valvular structure to form a pilot puncture in the host leaflet.

13. The delivery apparatus of claim 10, wherein the balloon, when positioned within a pilot puncture formed inside a host leaflet of a host valvular structure, is configured to expand the pilot puncture to form a leaflet opening.

14. A method comprising:
advancing a delivery apparatus comprising a steerable tube, over a guidewire, to a host valvular structure, the steerable tube comprising: two longitudinal slots defining a first tube portion and a second tube portion, a steerable tube distal portion extending distally from the longitudinal slots, and a slotted section which comprises a plurality of pairs of circumferential slots disposed along the second tube portion and defining a backbone opposite to the first tube portion;
bending the slotted section by axially moving the first tube portion and the second tube portion relative to each other; and
forming, with a piercing tip of the delivery apparatus, a pilot puncture within a host leaflet of the host valvular structure.

15. The method of claim 14, wherein the bending the slotted section comprises orienting the piercing tip towards the host leaflet.

16. The method of claim 14, wherein the forming the pilot puncture comprises distally advancing the piercing tip towards and through the host leaflet.

17. The method of claim 14, wherein the steerable tube is a steerable needle, and wherein the steerable tube distal portion comprises the piercing tip.

18. The method of claim 14, wherein the axially moving comprises proximally pulling the first tube portion relative to the second tube portion.

19. The method of claim 14, further comprising a dilator defining a dilator lumen, wherein the steerable tube is disposed inside the dilator lumen.

20. The method of claim 19, further comprising, subsequent to the forming the pilot puncture, passing the dilator through the pilot puncture, thereby expanding the pilot puncture.

21. The method of claim 20, further comprising a balloon catheter defining a balloon catheter lumen, and a balloon mounted on the balloon catheter and in fluid communication with the balloon catheter lumen.

22. The method of claim 21, further comprising, subsequent to the passing the dilator through the pilot puncture, positioning the balloon, in a radially deflated state thereof, within the pilot puncture.

23. The method of claim 22, further comprising, subsequent to the positioning the balloon, inflating the balloon to expand the pilot puncture and form a leaflet opening within the host leaflet.

24. A stabilized tissue modification system, comprising:
an anchor device comprising:
an anchor shaft defining an anchor shaft lumen;
a helical anchor head coupled to the anchor shaft, the helical anchor head defining an anchor channel in fluid communication with the anchor shaft lumen, the helical anchor head comprising: an anchor proximal end; and
at least one helical slot extending distally from the anchor proximal end, the at least one helical slot defining one or more helical turns, wherein a distal- most helical turn of the one or more helical turns defines a tip portion terminating at an anchor tip;
a balloon catheter defining a balloon catheter lumen; and
a hole-dilating balloon mounted on the balloon catheter and in fluid communication with the balloon catheter lumen, the hole-dilating balloon configured to transition between deflated and inflated states thereof;
wherein the anchor shaft is a flexible torque shaft configured to rotate around a central axis thereof, such that when the anchor shaft is rotated, the helical anchor head is configured to rotate therewith, and wherein the tip portion further comprises a cutting edge opposite to an inner side of the tip portion.

25. A steerable delivery system comprising: a handle; and a steerable catheter assembly extending distally from the handle and comprising: a steerable assembly lumen defining a steerable assembly central longitudinal axis; an inner tube comprising: an inner tube slotted portion defining an inner tube outer surface oriented away from the steerable assembly central longitudinal axis; and an inner tube distal end portion distal to the inner tube slotted portion; an outer tube disposed around the inner tube, the outer tube comprising: an outer tube slotted portion defining an outer tube inner surface oriented towards the steerable assembly central longitudinal axis; and an outer tube distal end portion distal to the inner tube slotted portion; and a pull-member comprising: a pull-ring portion affixed to the inner tube distal end portion and to the outer tube distal end portion; and at least one elongated pull-arm extending proximally from the pull-ring portion, wherein the at least one elongated pull-arm is disposed between the inner tube outer surface and the outer tube inner surface, and is axially slidable relative to the inner tube slotted portion and the outer tube slotted portion; wherein the at least one elongated pull-arm defines a circumferential width between arm circumferential ends that is greater than a radial thickness of the at least one elongated pull-arm; and wherein the at least one elongated pull-arm is configured to bend the steerable catheter assembly when the at least one elongated pull-arm is proximally pulled.
